(19) United States
(12) Patent Application Publication
(10) Pub. No.: US 2005/0152982 A1
(43) Pub. Date: Jul. 14, 2005

(54) Controlled release multiparticulates formed with dissolution enhancers

(75) Inventors: Leah E. Appel, Bend, OR (US); Roderick J. Ray, Bend, OR (US); David D. Newbold, Bend, OR (US); David K. Lyon, Bend, OR (US); James B. West, Bend, OR (US); Dwayne T. Friesen, Bend, OR (US); Scott B. McCray, Bend, OR (US); Marshall D. Crew, Bend, OR (US); Julian B. Lo, Old Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/003,853

(22) Filed: Dec. 3, 2004

Related U.S. Application Data
(60) Provisional application No. 60/527,319, filed on Dec. 4, 2003.

(51) Int. Cl. A61K 31/7052; A61K 9/22; A61K 9/14

(52) U.S. Cl. 424/489; 514/28

(57) ABSTRACT

Pharmaceutical compositions of crystalline azithromycin-containing multiparticulates having low concentrations of azithromycin ester degradants and exhibiting controlled release of the drug are achieved by inclusion of dissolution enhancers having low concentrations of acid and ester substituents.

### BACKGROUND OF THE INVENTION

Multiparticulates are well-known dosage forms that comprise a multiplicity of particles whose totality represents the intended therapeutically useful dose of a drug. When taken orally, multiparticulates generally disperse freely in the gastrointestinal tract, exit relatively rapidly and reproducibly from the stomach, maximize absorption, and minimize side effects. See, for example, Multiparticulate Oral Drug Delivery (Marcel Dekker, 1994), and Pharmaceutical Pelletization Technology (Marcel Dekker, 1989).

Azithromycin is the generic name for the drug 9a-aza-9a-methyl-9-deoxo-9a-homoerythromycin A, a broad-spectrum antimicrobial compound derived from erythromycin A. Accordingly, azithromycin and certain derivatives thereof are useful as antibiotics.

It is well known that oral dosing of azithromycin can result in the occurrence of adverse side effects such as cramping, diarrhea, nausea and vomiting. Such side effects are higher at higher doses than at lower doses. Multiparticulates are a known improved dosage form of azithromycin that permit higher oral dosing with relatively reduced side effects. See commonly owned U.S. Pat. No. 6,068,859. Such multiparticulates of azithromycin are particularly suitable for administration of single doses of the drug inasmuch as a relatively large amount of the drug can be delivered at a controlled rate over a relatively long period of time. A number of methods of formulating such azithromycin multiparticulates are disclosed in the '859 patent, including extrusion/spheronization, wax granulation, spray drying, and spray coating.

Multiparticulates are often used to provide controlled release of a drug. One problem when formulating a controlled release multiparticulate is setting the release rate of the drug. The release rate of the drug depends on a variety of factors, including the carriers used to form the multiparticulate and the amount of drug in the multiparticulate. It is desired to provide carriers for a multiparticulate which allow the release rate of the drug from the multiparticulate to be controlled over a wide range of release rates, so that the same matrix materials in different proportions may used to provide slow or fast drug release as desired. To achieve this result, the release rate of the drug should change significantly in response to relatively small changes in the proportions of the respective carriers in the multiparticulate.

The use of dissolution enhancers to control the release of drug from a wax or glyceride-based multiparticulate is known. U.S. Published Application No. 2001/0006650A1 discloses the formation of “solid solution” beadlets by a spray-congealing method comprising drug, a hydrophobic long chain fatty acid or ester and a surfactant. U.S. Pat. No. 6,013,280 discloses immediate release multiparticulate dosage forms comprising a polymeric solubilizing agent. Other disclosures of the use of dissolution enhancers with multiparticulates include U.S. Pat. Nos. 4,837,381, 4,880,634, 5,169,645, 5,571,533, 5,683,720, 5,849,223, 5,869,098, 6,013,280, 6,048,541, 6,086,920, 6,117,452 and 6,165,512. However, none of these references disclose the use of azithromycin as a suitable drug for inclusion in multiparticulates.

The inventors have discovered that certain processes used to form multiparticulates containing azithromycin and the use of certain excipients in such multiparticulates can lead to degradation of the azithromycin during and after the process of forming the multiparticulates. The degradation occurs by virtue of a chemical reaction of the azithromycin with the components of the carriers or excipients used in forming the multiparticulates, resulting in the formation of azithromycin esters. The prior art has not recognized this mechanism of azithromycin degradation, and no guidelines for the formation of azithromycin-containing multiparticulates or for selection of excipients that maintain azithromycin ester formation at acceptable levels have been suggested.

Thus, there is a need for an azithromycin multiparticulate that provides controlled release of the drug and that has acceptable concentrations of undesirable azithromycin esters.

### BRIEF SUMMARY OF THE INVENTION

The inventors have discovered that formation of azithromycin esters can be kept at acceptable levels by selection of a dissolution enhancer with certain properties, as detailed herein. Thus, the present invention provides a controlled release pharmaceutical composition of azithromycin multiparticulates having acceptable concentrations of azithromycin esters, comprising the drug, a pharmaceutically acceptable carrier and a pharmaceutically acceptable dissolution enhancer having a low concentration of carboxylic acid and ester substituents. The carrier has a melting point less than the melting point of azithromycin. In its broadest aspect, the pharmaceutical composition includes a dissolution enhancer that has a concentration of carboxylic acid and ester substituents of less than or equal to about 0.13 meq/g azithromycin and wherein the concentration of azithromycin esters is less than about 1 wt %. As used in the present invention, the term “about” means the specified value ±10% of the specified value.

All references to “acid and/or ester substituents” herein are intended to mean carboxylic acid, sulfonic acid, and phosphoric acid substituents or carboxylic acid ester, sulfonyl ester, or phosphate ester substituents, respectively.

In two related aspects, the present invention provides (1) a method of treating a patient in need of azithromycin therapy comprising administering a therapeutically effective amount of the inventive azithromycin multiparticulates and (2) azithromycin dosage forms comprising certain therapeutically effective amounts of the inventive azithromycin multiparticulates. The amount of azithromycin which is administered will necessarily be varied according to principles well known in the art, taking into account factors such as the severity of the disease or condition being treated and the size and age of the patient. In general, the drug is to be administered so that an effective dose is received, with the effective dose being determined from safe and efficacious ranges of administration already known for azithromycin.

The invention is particularly useful for administering relatively large amounts of azithromycin to a patient in a single-dose therapy. By “single dose therapy” is meant administering only one dose of azithromycin in the full course of therapy. The amount of azithromycin contained within the multiparticulate dosage form is preferably at least 250 mgA, and can be as high as 7 gA (“mgA” and “gA” mean milligrams and grams of active azithromycin in the dosage form, respectively). The amount contained in the dosage form is preferably about 1.5 to about 4 gA, more preferably about 1.5 to about 3 gA, and most preferably 1.8 to 2.2 gA. For small patients, e.g., children weighing about 30 kg or less, the multiparticulate dosage form can be scaled according to the weight of the patient; in one aspect, the dosage form contains about 30 to about 90 mgA/kg of patient body weight, preferably about 45 to about 75 mgA/kg, more preferably, about 60 mgA/kg. For veterinary applications, the dose may be adjusted to be outside these limits, depending upon the size of the animal.

An acceptable level of azithromycin ester formation is one which, during the time period beginning with formation of multiparticulates and continuing up until dosage, results in the formation of less than about 1 wt % azithromycin esters, meaning the weight of azithromycin esters relative to the total weight of azithromycin originally present in the multiparticulates, preferably less than about 0.5 wt %, more preferably less than about 0.2 wt %, and most preferably less than about 0.1 wt %.

The multiparticulates of the present invention are designed for controlled release of azithromycin after introduction to a use environment. As used herein, a “use environment” can be either the in vivo environment of the GI tract of a mammal such as a human, or the in vitro environment of a test solution. Exemplary test solutions include aqueous solutions at 37° C. comprising (1) 0.1 N HCl, simulating gastric fluid without enzymes; (2) 0.01 N HCl, simulating gastric fluid that avoids excessive acid degradation of azithromycin, and (3) 50 mM KH₂PO₄, adjusted to pH 6.8 using KOH or 50 mM Na₃PO₄, adjusted to pH 6.8 using NaOH, both of which simulate intestinal fluid without enzymes. The inventors have also found that for some formulations, an in vitro test solution comprising 100 mM Na₂HPO₄, adjusted to pH 6.0 using NaOH provides a discriminating means to differentiate among different formulations on the basis of dissolution profile. It has been determined that in vitro dissolution tests in such solutions provide a good indicator of in vivo performance and bioavailability. Further details of in vitro tests and test solutions are described herein.

Detailed guidelines on selection of dissolution enhancers, carriers, and processing conditions and their interrelationships are set forth in the Detailed Description of Preferred Embodiments below. Also according to the present invention, reaction rates for carriers and dissolution enhancers may be calculated so as to enable the practitioner to make an informed selection, following the general guideline that a carrier or dissolution enhancer exhibiting a slower rate of ester formation is desirable, while a carrier or dissolution enhancer exhibiting a faster rate of ester formation is undesirable.

### DETAILED DESCRIPTION OF PREFERRED EMBODIMENTS

The concentration of azithromycin esters present in the multiparticulate should be less than about 1 wt %; that is, the weight of azithromycin esters relative to the total azithromycin originally present in the multiparticulate should be less than about 1 wt %. Preferably, the concentration of azithromycin esters is less than about 0.5 wt %, more preferably less than about 0.2 wt %, and most preferably less than about 0.1 wt %.

Azithromycin esters may be formed during the multiparticulate-forming process, during other processing steps required for manufacture of the finished dosage form, or during storage following manufacture but prior to dosing. Since the azithromycin dosage forms may be stored for up to two years or even longer prior to dosing, it is preferred that the amount of azithromycin esters in the stored dosage form not exceed the above values prior to dosing.

The compositions of the present invention comprise a plurality of multiparticulates comprising azithromycin, a carrier and a dissolution enhancer, the multiparticulates exhibiting controlled release of the drug. The term “multiparticulates” is intended to embrace a dosage form comprising a multiplicity of particles whose totality represents the intended therapeutically useful dose of azithromycin. The particles generally are of a mean diameter from about 40 to about 3000 μm, preferably from about 50 to about 1000 μm, and most preferably from about 100 to about 300 μm. Preferably, the azithromycin makes up about 5 wt % to about 90 wt % of the total weight of the multiparticulate, more preferably about 10 wt % to about 80 wt %, even more preferably about 30 wt % to about 60 wt % of the total weight of the multiparticulates.

Multiparticulates represent a preferred embodiment because they are amenable to use in scaling dosage forms according to the weight of an individual patient in need of treatment by simply scaling the mass of particles in the dosage form to comport with the patient's weight. They are further advantageous since they allow the incorporation of a large quantity of drug into a simple dosage form such as a sachet that can be formulated into a slurry that can easily be consumed orally. Multiparticulates also have numerous therapeutic advantages over other dosage forms, especially when taken orally, including (1) improved dispersal in the gastrointestinal (GI) tract, (2) more uniform GI tract transit time, and (3) reduced inter- and intra-patient variability.

The invention also provides a method of treating a disease or condition amenable to treatment with azithromycin, comprising administering to an mammal, including a human, in need of such treatment multiparticulates containing an effective amount of azithromycin.

The amount of azithromycin which is administered will necessarily be varied according to principles well known in the art, taking into account factors such as the severity of the disease or condition being treated and the size and age of the patient. In general, the drug is to be administered so that an effective dose is received, with the effective dose being determined from safe and efficacious ranges of administration already known for azithromycin.

While the multiparticulates can have any shape and texture, it is preferred that they be spherical, with a smooth surface texture. These physical characteristics lead to excellent flow properties, improved “mouth feel,” ease of swallowing and ease of uniform coating, if required.

It has been found that under commonly used processing conditions azithromycin can react with certain excipients to form azithromycin esters. In particular, as described in more detail below, because the dissolution enhancer is typically more hydrophilic than the carrier, the solubility of azithromycin in the dissolution enhancer at the processing conditions is higher than in the carrier. As a result, the inventors have found that the concentration of acid and ester substituents on the dissolution enhancer should be low, e.g., less than about 0.13 meq/g azithromycin, to keep the amount of azithromycin esters in the composition at acceptable levels.

### Formation of Azithromycin Esters

Azithromycin esters can form either through direct esterification or transesterification of the hydroxyl substituents of azithromycin. By direct esterification is meant that an excipient having a carboxylic acid moiety can react with the hydroxyl substituents of azithromycin to form an azithromycin ester. By transesterification is meant that an excipient having an ester substituent can react with the hydroxyl groups, transferring the carboxylate of the carrier to azithromycin, also resulting in an azithromycin ester. Purposeful synthesis of azithromycin esters has shown that the esters typically form at the hydroxyl group attached to the 2′ carbon (C2′) of the desosamine ring; however esterification at the hydroxyls attached to the 4″ carbon on the cladinose ring (C4″) or the hydroxyls attached to the C6, C11, or C12 carbons on the macrolide ring may also occur in azithromycin formulations. An example of a transesterification reaction of azithromycin with a C₁₆ to C₂₂ fatty acid glyceryl triester is shown below.

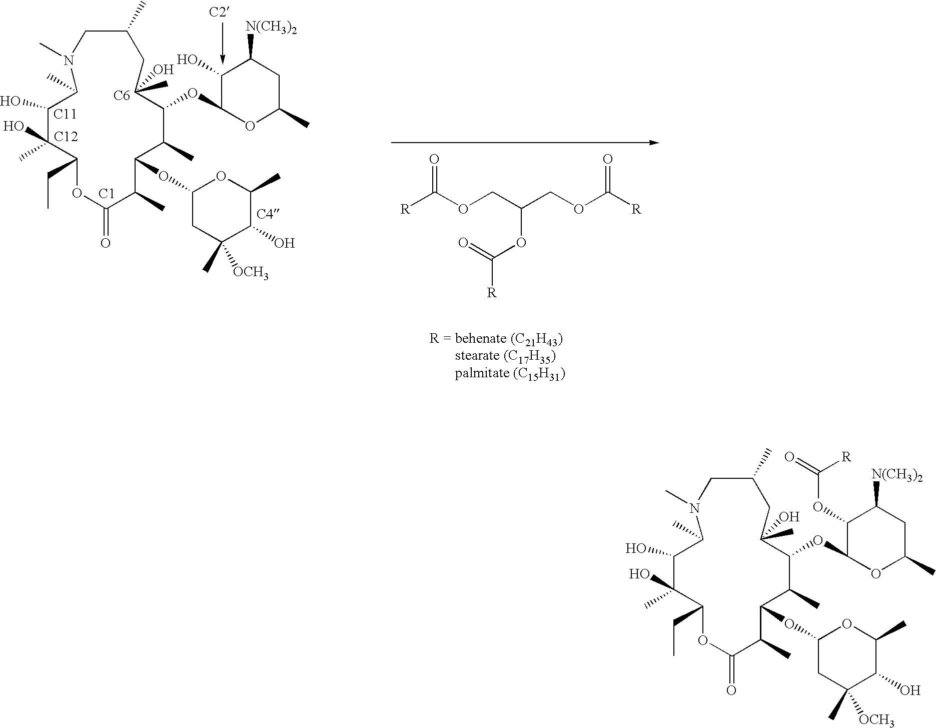

Typically in such reactions, one acid or one ester substituent on the excipient can each react with one molecule of azithromycin, although formation of two or more esters on a single molecule of azithromycin is possible. One convenient way to assess the potential for an excipient to react with azithromycin to form an azithromycin ester is the number of moles or equivalents of acid or ester substituents on the excipient per gram of azithromycin in the composition. For example, if an excipient has 0.13 milliequivalents (meq) of acid or ester substituents per gram of azithromycin in the composition and all of these acid or ester substituents reacted with azithromycin to form mono-substituted azithromycin esters, then 0.13 meq of azithromycin esters would form. Since the molecular weight of azithromycin is 749 g/mole, this means that 0.10 g of azithromycin would be converted to an azithromycin ester in the composition for every gram of azithromycin initially present in the composition. Thus, the concentration of azithromycin esters in the multiparticulates would be 10 wt %. However, it is unlikely that every acid and ester substituent in a composition will react to form azithromycin esters. Thus, to obtain a composition containing less than about 1 wt % azithromycin esters, the excipient should have no more than about 0.13 meq of acid and ester substituents per gram of azithromycin.

The rate of azithromycin ester formation Re in wt %/day for a given excipient at a temperature T (° C.) may be predicted using a zero-order reaction model, according to the following equation:
Rₑ=Cₑₛₜₑᵣₛ÷t (I)
where Cₑₛₜₑᵣₛ is the concentration of azithromycin esters formed (wt %) and t is time of contact between azithromycin and the excipient in days at temperature T.

One procedure for determining the reaction rate for forming azithromycin esters with the excipient is as follows. The excipient is heated to a constant temperature above its melting point and an equal weight of azithromycin is added to the molten excipient, thereby forming a suspension or solution of azithromycin in the molten excipient. Samples of the molten mixture are then periodically withdrawn and analyzed for formation of azithromycin esters using the procedures described below. The reaction rate for ester formation can then be determined using equation (I) above.

Alternatively, the excipient and azithromycin can be blended at a temperature below the melting temperature of the excipient and the blend stored at a convenient temperature, such as 50° C. Samples of the blend can be periodically removed and analyzed for azithromycin esters, as described below. The rate of ester formation can then be determined using Equation (I) above.

A number of methods well known in the art can be used to determine the concentration of azithromycin esters in multiparticulates. An exemplary method is by high performance liquid chromatography/mass spectrometry (LC/MS) analysis. In this method, the azithromycin and any azithromycin esters are extracted from the multiparticulates using an appropriate solvent, such as methanol or isopropyl alcohol. The extraction solvent may then be filtered with a 0.45 μm nylon syringe filter to remove any particles present in the solvent. The various species present in the extraction solvent can then be separated by high performance liquid chromatography (HPLC) using procedures well known in the art. A mass spectrometer is used to detect species, with the concentrations of azithromycin and azithromycin esters being calculated from the mass spectrometer peak areas based on either an internal or external azithromycin control. Preferably, if authentic standards of the esters have been synthesized, external references to the azithromycin esters may be used. The azithromycin ester value is then reported as a percentage of the total azithromycin in the sample.

To satisfy a total azithromycin esters content of less than about 1 wt %, the rate of total azithromycin esters formation should be
Rₑ≦3.6×10⁷·e^{-70701/(T+273)},
where T is the temperature in ° C.

To satisfy the preferred total azithromycin esters content of less than about 0.5 wt %, rate of total azithromycin esters formation should be
Rₑ≦1.8×10⁷·e^{-7070/(T+273)}.

To satisfy the more preferred total azithromycin esters content of less than about 0.2 wt %, the rate of total azithromycin esters formation should be
Rₑ≦7.2×10⁶·e^{-7070/(T+273)}.

To satisfy the most preferred total azithromycin esters content of less than about 0.1 wt %, the rate of total azithromycin esters formation should be
Rₑ≦3.6×10⁶·e^{-7070/(T+273)}.

### Dissolution Enhancers

The multiparticulate compositions of the present invention include a pharmaceutically acceptable dissolution enhancer. By “pharmaceutically acceptable” is meant the dissolution enhancer must be compatible with the other ingredients of the composition, and not deleterious to the recipient thereof. By “dissolution enhancer” is meant an excipient that when included in the multiparticulates, results in a faster rate of release of azithromycin than that provided by a control multiparticulate containing the same amount of azithromycin but does not contain the dissolution enhancer. Generally, the mass of dissolution enhancer present in the multiparticulate is less than the mass of carrier present in the multiparticulate. The amount of dissolution enhancer present in the multiparticulate can range from about 0.1 to about 30 wt %, preferably from about 0.1 to about 15 wt %, based on the total mass of the multiparticulate.

The inventors have found that the azithromycin present in the multiparticulate is particularly reactive with dissolution enhancers. As a result, the concentration of acid and ester substituents on the dissolution enhancer must be kept low to keep the formation of azithromycin esters at acceptably low levels.

Without wishing to be bound by any theory or mode of action, it is believed that azithromycin is more reactive with dissolution enhancers for the following reasons. Dissolution enhancers tend to be more hydrophilic than carriers, often being readily soluble or dispersible in water. As a result, the solubility of azithromycin in the dissolution enhancer at processing conditions is often high. The reactivity of dissolved azithromycin is much higher than that of crystalline azithromycin. In crystalline azithromycin, the azithromycin molecules are locked into a rigid three-dimensional structure that is at a low thermodynamic energy state. Removal of an azithromycin molecule from this crystal structure, for example, to react with an excipient, will therefore take a considerable amount of energy. In addition, crystal forces reduce the mobility of the azithromycin molecules in the crystal structure. The result is that the rate of reaction of azithromycin with acid and ester substituents on an excipient is significantly reduced in crystalline azithromycin when compared to formulations containing amorphous or dissolved azithromycin.

A convenient way to assess the potential for azithromycin to react with a dissolution enhancer to form azithromycin esters is to ascertain the dissolution enhancer's degree of acid/ester substitution. This can be determined by dividing the number of acid and ester substituents on each dissolution enhancer molecule by its molecular weight, yielding the number of acid and ester substituents per gram of each dissolution enhancer molecule. As many suitable dissolution enhancers are actually mixtures of several specific molecule types, average values of numbers of substituents and molecular weight may be used in these calculations. The concentration of acid and ester substituents per gram of azithromycin in the composition may then be determined by multiplying this number by the mass of dissolution enhancer in the composition and dividing by the mass of azithromycin in the composition. For example, polyoxyethylene sorbitan fatty acid esters, such as polysorbate 80 (also known as polyoxyethylene 20 sorbitan monooleate), having the structure 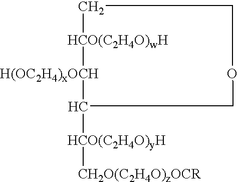
where w+x+y+z 20, and R is oleate, has a molecular weight of 1310 g/mol and one ester substituent per mole. Thus, the ester substituents concentration per gram of the dissolution enhancer polysorbate 80 is 1÷1310 g or 0.0008 eq/g dissolution enhancer, or 0.8 meq/g dissolution enhancer. If a multiparticulate is formed containing 50 wt % azithromycin and 5 wt % polysorbate 80, the ester substituent concentration per gram of azithromycin would be
0.8 meq/g×5/50=0.08 meq/g azithromycin.
The above calculation can be used to calculate the concentration of acid and ester substituents on any dissolution enhancer candidate.

However, in most cases, the dissolution enhancer candidate is not available in pure form, and may constitute a mixture of several primary molecular types as well as small amounts of impurities or degradation products that could contain acids or esters. In addition, many dissolution enhancer candidates are natural products or are derived from natural products that may contain a wide range of compounds, making the above calculations extremely difficult, if not impossible. For these reasons, the inventors have found that the degree of acid/ester substitution on such materials can often most easily be estimated by using the Saponification Number or Saponification Value of the dissolution enhancer. The Saponification Number is the number of milligrams of potassium hydroxide required to neutralize or hydrolyze any acid or ester substituents present in 1 gram of the material. Measurement of the Saponification Number is a standard way to characterize many commercially available dissolution enhancer excipients and the manufacturer often provides the excipient's Saponification Number. The Saponification Number will not only account for acid and ester substituents present on the dissolution enhancer itself, but also for any such substituents present due to impurities or degradation products in the dissolution enhancer. Thus, the Saponification Number will often provide a more accurate measure of the degree of acid/ester substitution in the dissolution enhancer.

One procedure for determining the Saponification Number of a candidate dissolution enhancer is as follows. A potassium hydroxide solution is prepared by first adding 5 to 10 g of potassium hydroxide to one liter of 95% ethanol and boiling the mixture under a reflux condenser for about an hour. The ethanol is then distilled and cooled to below 15.5° C. While keeping the distilled ethanol below this temperature, 40 g of potassium hydroxide is dissolved in the ethanol, forming the alkaline reagent. A 4 to 5 g sample of the dissolution enhancer is then added to a flask equipped with a refluxing condenser. A 50 mL sample of the alkaline reagent is then added to the flask and the mixture is boiled under refluxing conditions until saponification is complete, generally, about an hour. The solution is then cooled and 1 ml of phenolphthalein solution (1% in 95% ethanol) is added to the mixture and the mixture titrated with 0.5 N HCl until the pink color just disappears. The Saponification Number in mg of potassium hydroxide per g material is then calculated from the following equation:
Saponification Number=[28.05×(B−S)]÷weight of sample
where B is the number of mL of HCl required to titrate a blank sample (a sample containing no dissolution enhancer) and S is the number of mL of HCl required to titrate the sample. Further details of such a method for determining the Saponification Number of a material is given in Welcher, Standard Methods of Chemical Analysis (1975). The American Society for Testing and Materials (ASTM) also has established several tests for determining the Saponification Number for various materials, such as ASTM D1387-89, D94-00, and D558-95. These methods may also be appropriate for determining the Saponification Number for a potential dissolution enhancer.

For some dissolution enhancers, the processing conditions used to form the multiparticulates (e.g., high temperature) may result in a change in the chemical structure of the dissolution enhancer, possibly leading to the formation of acid and/or ester substituents, e.g., by oxidation. Thus, the Saponification Number of a dissolution enhancer should be measured after it has been exposed to the processing conditions anticipated for forming the multiparticulates. In this way, potential degradation products from the dissolution enhancer that may result in the formation of azithromycin esters can be accounted for.

The degree of acid and ester substitution of a dissolution enhancer material can be calculated from the Saponification Number as follows. Dividing the Saponification Number by the molecular weight of potassium hydroxide, 56.11 g/mol, results in the number of millimoles of potassium hydroxide required to neutralize or hydrolyze any acid or ester substituents present in one gram of the dissolution enhancer. Since one mole of potassium hydroxide will neutralize one equivalent of acid or ester substituents, dividing the Saponification Number by the molecular weight of potassium hydroxide will yield the number of milliequivalents (meq) of acid or ester substituents present in one gram of dissolution enhancer.

For example, polyoxyethylene sorbitan fatty acid esters can be obtained with a Saponification Number of 55, as specified by the manufacturer. Thus, the degree of acid/ester substitution per gram of dissolution enhancer or its acid/ester concentration is
55÷56.11=0.98 meq/g dissolution enhancer.
Using the above example of a composition with 50 wt % azithromycin and 5 wt % polysorbate 80, the theoretical concentration of esters formed per gram of azithromycin if all of the azithromycin reacted would be
0.98 meq/g×5/50=0.1 meq/g azithromycin.

From the standpoint of reactivity to form azithromycin esters, the dissolution enhancers preferably have a concentration of acid/ester substituents of less than about 0.13 meq/g azithromycin present in the composition. Preferably, the dissolution enhancer has a concentration of acid/ester substituents of less than about 0.10 meq/g azithromycin, more preferably less than about 0.02 meq/g azithromycin, even more preferably less than about 0.01 meq/g, and most preferably less than about 0.002 meq/g.

In addition to having low concentrations of acid and ester substituents, the dissolution enhancer should generally be hydrophilic, such that the rate of release of azithromycin from the multiparticulate increases as the concentration of dissolution enhancer in the multiparticulate increases. Preferred classes of materials are surfactants that can promote solubilization of other excipients in the composition.

Examples of dissolution enhancers that may be included in the composition include surfactants, such as poloxamers (polyoxyethylene polyoxypropylene copolymers, such as poloxamer 188, poloxamer 237, poloxamer 338, and poloxamer 407), such as the PLURONIC® and LUTROL® series (BASF Corporation, Mt. Olive, N.J.), polyoxyethylene alkyl esters and ethers, such as BRIJ (ICI Surfactants, Everberg, Belgium) and CHREMOPHOR A (BASF Corporation), polyoxyethylene castor oil derivatives, such as CHREMOPHOR RH40, polyoxyethylene sorbitan fatty acid esters, such as TWEEN 80 (ICI Surfactants) and CAPMUL POE-O (Karlshamns USA, Columbus, Ohio.), sorbitan esters, such as CAPMUL-O and SPAN 80 (ICI Surfactants), and alkyl sulfates, such as sodium lauryl sulfate; sugars such as glucose, sucrose, xylitol, sorbitol, and maltitol; alcohols, such as stearyl alcohol, cetyl alcohol, and low molecular weight (i.e., less than about 10,000 daltons) polyethylene glycol; salts such as sodium chloride, potassium chloride, lithium chloride, calcium chloride, magnesium chloride, sodium sulfate, potassium sulfate, sodium carbonate, magnesium sulfate, and potassium phosphate; amino acids such as alanine and glycine; ether-substituted cellulosics, such as hydroxypropyl cellulose and hydroxypropyl methyl cellulose; and mixtures thereof. Preferably, the dissolution enhancer is a surfactant, and most preferably, the dissolution enhancer is a poloxamer.

While not wishing to be bound by any particular theory or mechanism, it is believed that the dissolution enhancers present in the multiparticulates affect the rate at which the aqueous use environment penetrates the multiparticulate, thus affecting the rate at which azithromycin is released. In addition, such dissolution enhancers may enhance the azithromycin release rate by aiding in the aqueous dissolution of the carrier itself, often by solubilizing the carrier in micelles.

Note that some of the above dissolution enhancers may be suitable in one multiparticulate formulation, but not in another. For example, use of a polyoxyethylene sorbitan fatty acid ester dissolution enhancer with a concentration of acid and ester substituents of 0.8 meq/g dissolution enhancer is suitable for use in a composition comprising 50 wt % azithromycin and 5 wt % of the dissolution enhancer, as calculated above (0.8×5/50=0.08 meq/g azithromycin). However, if a faster rate of release of azithromycin was required and the concentration of the polyoxyethylene sorbitan fatty acid ester dissolution enhancer had to be increased to 10 wt %, the concentration of acid and ester substituents would be 0.16 meq/g azithromycin (0.8×10/50=0.16 meq/g), exceeding the target value of less than about 0.13 meq/g.

A preferred class of dissolution enhancers is poloxamers. These materials are a series of closely related block copolymers of ethylene oxide and propylene oxide that have no acid or ester substituents. This being the case, large amounts of poloxamers—as much as 30 wt % or more—can be used in a multiparticulate formulation and still meet the target value of less than about 0.13 meq/g of azithromycin. The inventors have also found that use of poloxamers as a dissolution enhancer allows for precise control of the rate of release of azithromycin from the multiparticulate. This is disclosed more fully in commonly assigned U.S. patent application Ser. No. ______ (“Multiparticulate Crystalline Drug Compositions Having Controlled Release Profiles,” Attorney Docket No. PC25020), filed concurrently herewith.

While the specific dissolution enhancers disclosed herein are suitable for use in the present invention, it should be understood that blends and mixtures of such dissolution enhancers may also be suitable.

### Azithromycin

The multiparticulates of the present invention comprise azithromycin. Preferably, the azithromycin makes up from about 5 wt % to about 90 wt % of the total weight of the multiparticulate, more preferably from about 10 wt % to about 80 wt %, and even more preferably from about 30 wt % to about 60 wt % of the total weight of the multiparticulates.

As used herein, “azithromycin” means all amorphous and crystalline forms of azithromycin including all polymorphs, isomorphs, pseudomorphs, clathrates, salts, solvates and hydrates of azithromycin, as well as anhydrous azithromycin. Reference to azithromycin in terms of therapeutic amounts or in release rates in the claims is to active azithromycin, i.e., the non-salt, non-hydrated azalide molecule having a molecular weight of 749 g/mole.

Preferably, the azithromycin of the present invention is azithromycin dihydrate, which is disclosed in U.S. Pat. No. 6,268,489.

In alternate embodiments of the present invention, the azithromycin comprises a non-dihydrate azithromycin, a mixture of non-dihydrate azithromycins, or a mixture of azithromycin dihydrate and non-dihydrate azithromycins. Examples of suitable non-dihydrate azithromycins include, but are not limited to, alternate crystalline forms B, D, E, F, G, H, J, M, N, O, P, Q and R.

Azithromycin also occurs as Family I and Family II isomorphs, which are hydrates and/or solvates of azithromycin. The solvent molecules in the cavities have a tendency to exchange between solvent and water under specific conditions. Therefore, the solvent/water content of the isomorphs may vary to a certain extent.

Azithromycin form B, a hygroscopic hydrate of azithromycin, is disclosed in U.S. Pat. No. 4,474,768.

Azithromycin forms D, E, F, G, H, J, M, N, O, P, Q and R are disclosed in commonly owned U.S. Patent Publication No. 20030162730, published Aug. 28, 2003.

Forms B, F, G, H, J, M, N, O, and P belong to Family I azithromycin and have a monoclinic P2₁ space group with cell dimensions of a=16.3±0.3 Å, b=16.2±0.3 Å, c=18.4±0.3 Å and beta=109±2°.

Form F azithromycin is an azithromycin ethanol solvate of the formula C₃₈H₇₂N₂O₁₂.H₂O.0.5C₂H₅OH in the single crystal structure and is an azithromycin monohydrate hemi-ethanol solvate. Form F is further characterized as containing 2-5 wt % water and 1-4 wt % ethanol by weight in powder samples. The single crystal of form F is crystallized in a monoclinic space group, P2₁, with the asymmetric unit containing two azithromycin molecules, two water molecules, and one ethanol molecule, as a monohydrate/hemi-ethanolate. It is isomorphic to all Family I azithromycin crystalline forms. The theoretical water and ethanol contents are 2.3 and 2.9 wt %, respectively.

Form G azithromycin has the formula C₃₈H₇₂N₂O₁₂.1.5H₂O in the single crystal structure and is an azithromycin sesquihydrate. Form G is further characterized as containing 2.5-6 wt % water and <1 wt % organic solvent(s) by weight in powder samples. The single crystal structure of form G consists of two azithromycin molecules and three water molecules per asymmetric unit, corresponding to a sesquihydrate with a theoretical water content of 3.5 wt %. The water content of powder samples of form G ranges from about 2.5 to about 6 wt %. The total residual organic solvent is less than 1 wt % of the corresponding solvent used for crystallization.

Form H azithromycin has the formula C₃₈H₇₂N₂O₁₂.H₂O.0.5C₃H₈O₂ and may be characterized as an azithromycin monohydrate hemi-1,2 propanediol solvate. Form H is a monohydrate/hemi-propylene glycol solvate of azithromycin free base.

Form J azithromycin has the formula C₃₈H₇₂N₂O₁₂.H₂O.0.5C₃H₇OH in the single crystal structure, and is an azithromycin monohydrate hemi-n-propanol solvate. Form J is further characterized as containing 2-5 wt % water and 1-5 wt % n-propanol by weight in powder samples. The calculated solvent content is about 3.8 wt % n-propanol and about 2.3 wt % water.

Form M azithromycin has the formula C₃₈H₇₂N₂O₁₂.H₂O.0.5C₃H₇OH, and is an azithromycin monohydrate hemi-isopropanol solvate. Form M is further characterized as containing 2-5 wt % water and 1-4 wt % 2-propanol by weight in powder samples. The single crystal structure of form M would be a monohydrate/hemi-isopropranolate.

Form N azithromycin is a mixture of isomorphs of Family I. The mixture may contain variable percentages of isomorphs F, G, H, J, M and others, and variable amounts of water and organic solvents, such as ethanol, isopropanol, n-propanol, propylene glycol, acetone, acetonitrile, butanol, pentanol, etc. The weight percent of water can range from 1-5.3 wt % and the total weight percent of organic solvents can be 2-5 wt % with each solvent making up 0.5-4 wt %.

Form O azithromycin has the formula C₃₈H₇₂N₂O₁₂.0.5H₂O.0.5C₄H₉OH, and is a hemihydrate hemi-n-butanol solvate of azithromycin free base by single crystal structural data.

Form P azithromycin has the formula C₃₈H₇₂N₂O₁₂.H₂O.0.5C₅H₁₂O and is an azithromycin monohydrate hemi-n-pentanol solvate.

Form Q is distinct from Families I and II, has the formula C₃₈H₇₂N₂O₁₂.H₂O.0.5C₄H₈O and is an azithromycin monohydrate hemi-tetrahydrofuran (THF) solvate. It contains about 4% water and about 4.5 wt % THF.

Forms D, E and R belong to Family II azithromycin and contain an orthorhombic P2₁, 2₁2₁ space group with cell dimensions of a=8.9±0.4 Å, b=12.3±0.5 Å and c=45.8±0.5 Å.

Form D azithromycin has the formula C₃₈H₇₂N₂O₁₂.H₂O.C₆H₁₂ in its single crystal structure, and is an azithromycin monohydrate monocyclohexane solvate. Form D is further characterized as containing 2-6 wt % water and 3-12 wt % cyclohexane by weight in powder samples. From single crystal data, the calculated water and cyclohexane content of form D is 2.1 and 9.9 wt %, respectively.

Form E azithromycin has the formula C₃₈H₇₂N₂O₁₂.H₂O.C₄H₈O and is an azithromycin monohydrate mono-THF solvate by single crystal analysis.

Form R azithromycin has the formula C₃₈H₇₂N₂O₁₂.H₂O.C₅H₁₂O and is an azithromycin monohydrate mono-methyl tert-butyl ether solvate. Form R has a theoretical water content of 2.1 wt % and a theoretical methyl tert-butyl ether content of 10.3 wt %.

Other examples of non-dihydrate azithromycin include, but are not limited to, an ethanol solvate of azithromycin or an isopropanol solvate of azithromycin. Examples of such ethanol and isopropanol solvates of azithromycin are disclosed in U.S. Pat. Nos. 6,365,574 and 6,245,903 and U.S. Patent Application Publication No. 20030162730, published Aug. 28, 2003.

Additional examples of non-dihydrate azithromycin include, but are not limited to, azithromycin monohydrate as disclosed in U.S. Patent Application Publication Nos. 20010047089, published Nov. 29, 2001, and 20020111318, published Aug. 15, 2002, as well as International Application Publication Nos. WO 01/00640, WO 01/49697, WO 02/10181 and WO 02/42315.

Further examples of non-dihydrate azithromycin include, but are not limited to, anhydrous azithromycin as disclosed in U.S. Patent Application Publication No. 20030139583, published Jul. 24, 2003, and U.S. Pat. No. 6,528,492.

Examples of suitable azithromycin salts include, but are not limited to, the azithromycin salts as disclosed in U.S. Pat. No. 4,474,768.

Preferably, at least 70 wt % of the azithromycin in the multiparticulate is crystalline. The degree of azithromycin crystallinity in the multiparticulates can be “substantially crystalline,” meaning that the amount of crystalline azithromycin in the multiparticulates is at least about 80%, “almost completely crystalline,” meaning that the amount of crystalline azithromycin is at least about 90%, or “essentially crystalline,” meaning that the amount of crystalline azithromycin in the multiparticulates is at least 95%. Preferably, the azithromycin is substantially in the crystalline dihydrate form, meaning that at least 80% of the azithromycin is in that crystalline form.

The crystallinity of azithromycin in the multiparticulates may be determined using Powder X-Ray Diffraction (PXRD) analysis. In an exemplary procedure, PXRD analysis may be performed on a Bruker AXS D8 Advance diffractometer. In this analysis, samples of about 500 mg are packed in Lucite sample cups and the sample surface smoothed using a glass microscope slide to provide a consistently smooth sample surface that is level with the top of the sample cup. Samples are spun in the φ plane at a rate of 30 rpm to minimize crystal orientation effects. The X-ray source (S/B KCu_{α}, λ=1.54 Å) is operated at a voltage of 45 kV and a current of 40 mA. Data for each sample are collected over a period of from about 20 to about 60 minutes in continuous detector scan mode at a scan speed of 12 seconds/step and a step size of 0.02°/step. Diffractograms are collected over the 2θ range of 10° to 16°.

The crystallinity of the test sample is determined by comparison with calibration standards as follows. The calibration standards consist of physical mixtures of 20 wt %/80 wt % azithromycin/carrier, and 80 wt %/20 wt % azithromycin/carrier. Each physical mixture is blended together 15 minutes on a Turbula mixer. Using the instrument software, the area under the diffractogram curve is integrated over the 20 range of 10° to 16° using a linear baseline. This integration range includes as many azithromycin-specific peaks as possible while excluding carrier-related peaks. In addition, the large azithromycin-specific peak at approximately 10° 2θ is omitted due to the large scan-to-scan variability in its integrated area. A linear calibration curve of percent crystalline azithromycin versus the area under the diffractogram curve is generated from the calibration standards. The crystallinity of the test sample is then determined using these calibration results and the area under the curve for the test sample. Results are reported as a mean percent azithromycin crystallinity (by crystal mass). As mentioned above, crystalline azithromycin is preferred since it is more chemically and physically stable than the amorphous form or dissolved azithromycin.

### Carriers

The multiparticulates comprise a pharmaceutically acceptable carrier. By “pharmaceutically acceptable” is meant the carrier must be compatible with the other ingredients of the composition, and not deleterious to the recipient thereof. The carrier functions as a matrix for the multiparticulate or to affect the rate of release of azithromycin from the multiparticulate, or both. Carriers will generally make up from about 10 wt % to about 95 wt % of the multiparticulate, preferably from about 20 wt % to about 90 wt %, and more preferably from about 40 wt % to about 70 wt % of the multiparticulate, based on the total mass of the multiparticulate. The carrier is preferably solid at temperatures of about 40° C. The inventors have found that if the carrier is not a solid at 40° C., there can be changes in the physical characteristics of the composition over time, especially when stored at elevated temperatures, such as at 40° C. Thus, it is preferred that the carrier be a solid at a temperature of about 50° C., and more preferably at about 60° C. It is also preferred that the carrier have a melting point that is less then the melting point of azithromycin. For example, azithromycin dihydrate has a melting point of 113° C. to 115° C. Thus, when azithromycin dihydrate is used in the multiparticulates of the present invention, it is preferred that the carrier have a melting point that is less than about 113° C. Preferably, the carrier is different than the dissolution enhancer.

Carriers can be classified into four general categories (1) non-reactive; (2) low reactivity; (3) moderate reactivity; and (4) highly reactive relative to their tendency to form azithromycin esters.

Non-reactive carriers generally have no acid or ester substituents and are free from impurities that contain acids or esters. Generally, non-reactive materials will have an acid/ester concentration of less than 0.0001 meq/g carrier. Non-reactive carriers are very rare since most materials contain small amounts of impurities. Non-reactive carriers must therefore be highly purified. In addition, non-reactive carriers are often hydrocarbons, since the presence of other elements in the carrier can lead to acid or ester impurities. The rate of formation of azithromycin esters for non-reactive carriers is essentially zero, with no azithromycin esters forming under the conditions described above for determining the azithromycin reaction rate with a carrier. Examples of non-reactive carriers include highly purified forms of the following hydrocarbons: synthetic wax, microcrystalline wax, and paraffin wax.

Low reactivity carriers also do not have acid or ester substituents, but often contain small amounts of impurities or degradation products that contain acid or ester substituents. Generally, low reactivity carriers have an acid/ester concentration of less than about 0.1 meq/g of carrier. Generally, low reactivity carriers will have a rate for formation of azithromycin esters of less than about 0.005 wt %/day when measured at 100° C. Examples of low reactivity carriers include long-chain alcohols, such as stearyl alcohol, cetyl alcohol, and polyethylene glycol; and ether-substituted cellulosics, such as microcrystalline cellulose, hydroxypropyl cellulose, hydroxypropyl methyl cellulose, and ethylcellulose.

Moderate reactivity carriers often contain acid or ester substituents, but relatively few as compared to the molecular weight of the carrier. Generally, moderate reactivity carriers have an acid/ester concentration of about 0.1 to about 3.5 meq/g of carrier. Examples include long-chain fatty acid esters, such as glyceryl monooleate, glyceryl monostearate, glyceryl palmitostearate, polyethoxylated castor oil derivatives, glyceryl dibehenate, and mixtures of mono-, di-, and tri-alkyl glycerides, including mixtures of glyceryl mono-, di-, and tribehenate, glyceryl tristearate, glyceryl tripalmitate and hydrogenated vegetable oils; and waxes, such as Carnauba wax and white and yellow beeswax.

Highly reactive carriers usually have several acid or ester substituents or low molecular weights. Generally, highly reactive carriers have an acid/ester concentration of more than about 3.5 meq/g of carrier and have a rate of formation of azithromycin esters of more than about 40 wt %/day at 100° C. Examples include carboxylic acids such as stearic acid, benzoic acid, and citric acid. Generally, the acid/ester concentration on highly reactive carriers is so high that if these carriers come into direct contact with azithromycin in the formulation, unacceptably high concentrations of azithromycin esters may form during processing or storage of the composition. Thus, such highly reactive carriers are preferably only used in combination with a carrier with lower reactivity so that the total amount of acid and ester groups on the carrier used in the multiparticulate is low. Preferably the carrier is selected from a non-reactive carrier, a low reactivity carrier, or a moderate reactivity carrier.

Preferred carriers suitable for use in the multiparticulates of the present invention include waxes, such as synthetic wax, microcrystalline wax, paraffin wax, carnauba wax, and beeswax; glycerides, such as glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate; long-chain alcohols, such as stearyl alcohol, cetyl alcohol, and polyethylene glycol; and mixtures thereof.

In one embodiment, the multiparticulates comprise about 20 to about 75 wt % azithromycin; about 25 to about 80 wt % of a carrier; and about 0.1 to about 30 wt % of a dissolution enhancer based on the total mass of the multiparticulate.

In another embodiment, the multiparticulates comprise about 35 wt % to about 55 wt % azithromycin; about 40 to about 65 wt % of a carrier selected from waxes, such as synthetic wax, microcrystalline wax, paraffin wax, Carnauba wax, and beeswax; glycerides, such as glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate and mixtures thereof; and about 0.1 to about 15 wt % of a dissolution-enhancer selected from the group comprising surfactants, such as poloxamers, polysorbates, polyoxyethylene alkyl esters, polyoxyethylene alkyl ethers, polyoxyethylene castor oil derivatives, polyoxyethylene sorbitan fatty acid esters, sorbitan esters, and sodium lauryl sulfate; sugars, such as glucose, sucrose, xylitol, sorbitol and maltitol; alcohols, such as stearyl alcohol, cetyl alcohol and polyethylene glycol; salts, such as sodium chloride, potassium chloride, lithium chloride, calcium chloride, magnesium chloride, sodium sulfate, potassium sulfate, sodium carbonate, magnesium sulfate, and potassium phosphate; amino acids, such as alanine and glycine; and mixtures thereof.

In another embodiment comprises from about 45 to about 55 wt % azithromycin; the same wt % range of a carrier; and from about 0.1 to about 5 wt % of a surfactant dissolution enhancer.

In yet another embodiment, the multiparticulates of the present invention comprise (a) azithromycin; (b) a glyceride carrier having at least one alkylate substituent of 16 or more carbon atoms; and (c) a poloxamer dissolution enhancer. At least 70 wt % of the drug in the multiparticulates is crystalline. The choice of these particular carrier excipients allows for precise control of the release rate of the azithromycin over a wide range of release rates. Small changes in the relative amounts of the glyceride carrier and the poloxamer result in large changes in the release rate of the drug. This allows the release rate of the drug from the multiparticulate to be precisely controlled by selecting the proper ratio of drug, glyceride carrier and poloxamer. These materials have the further advantage of releasing nearly all of the drug from the multiparticulate. Such multiparticulates are disclosed more fully in commonly assigned U.S. patent application Ser. No. ______ (“Multiparticulate Crystalline Drug Compositions Having Controlled Release Profiles,” Attorney Docket No. PC25020), filed concurrently herewith.

In one aspect, the multiparticulates are in the form of a non-disintegrating matrix. By “non-disintegrating matrix” is meant that at least a portion of the carrier does not dissolve or disintegrate after introduction of the multiparticulate to an aqueous use environment. In such cases, the azithromycin and optionally the dissolution enhancer, are released from the multiparticulate by dissolution. At least a portion of the carrier does not dissolve or disintegrate and is excreted when the use environment is in vivo, or remains suspended in a test solution when the use environment is in vitro. In this aspect, it is preferred that the carrier have a low solubility in the aqueous use environment. Preferably, the solubility of the carrier in the aqueous use environment is less than about 1 mg/mL, more preferably less than about 0.1 mg/mL, and most preferably less than about 0.01 mg/mL. Examples of suitable low-solubility carriers include waxes, such as synthetic wax, microcrystalline wax, paraffin wax, carnauba wax, and beeswax; glycerides, such as glyceryl monooleate, glyceryl monostearate, glyceryl palmitostearate, glyceryl mono-, di- or tribehenates, glyceryl tristearate, glyceryl tripalmitate and mixtures thereof.

### Processes for Forming Multiparticulates

Preferred processes to form the controlled release multiparticulates include thermal-based processes such as melt- and spray-congealing; liquid-based processes, such as extrusion spheronization, wet granulation, spray-coating and spray-drying and other granulation processes, such as dry granulation and melt granulation. Suitable thermal-based processes are disclosed in further detail in commonly assigned U.S. patent application Ser. No. ______ (“Improved Azithromycin Multiparticulate Dosage Forms by Melt-Congeal Processes,” Attorney Docket No. PC25015) filed concurrently herewith. Suitable liquid-based processes are disclosed in further detail in commonly assigned U.S. patent application Ser. No. ______ (“Improved Azithromycin Multiparticulate Dosage Forms by Liquid-Based Processes,” Attorney Docket No. PC25018) filed concurrently herewith.

In one aspect, the multiparticulates are made by a melt-congeal process comprising the steps (a) forming a molten mixture comprising azithromycin, a pharmaceutically acceptable carrier, and a dissolution enhancer, (b) delivering the molten mixture of step (a) to an atomizing means to form droplets from the molten mixture, and (c) congealing the droplets from step (b) to form multiparticulates.

The azithromycin in the molten mixture may be dissolved in the molten mixture, may be a suspension of crystalline azithromycin distributed in the molten mixture, or any combination of such states or those states that are in between. Preferably, the molten mixture is a homogeneous suspension of crystalline azithromycin in the molten carrier where the fraction of azithromycin that melts or dissolves in the molten carrier is kept relatively low. Preferably less than about 30 wt % of the total azithromycin melts or dissolves in the molten carrier. It is preferred that the azithromycin be present as the crystalline dihydrate.

Thus, “molten mixture” as used herein refers to a mixture of azithromycin and carrier heated sufficiently that the mixture becomes sufficiently fluid that the mixture may be formed into droplets or atomized. Atomization of the molten mixture may be carried out using any of the atomization methods described below. Generally, the mixture is molten in the sense that it will flow when subjected to one or more forces such as pressure, shear, and centrifugal force, such as that exerted by a centrifugal or spinning-disk atomizer. Thus, the azithromycin/carrier mixture may be considered “molten” when the mixture, as a whole, is sufficiently fluid that it may be atomized. Generally, a mixture is sufficiently fluid for atomization when the viscosity of the molten mixture is less than about 20,000 cp, preferably less than about 15,000 cp, and most preferably less than about 10,000 cp. Often, the mixture becomes molten when the mixture is heated above the melting point of one or more of the carrier components, in cases where the carrier is sufficiently crystalline to have a relatively sharp melting point; or, when the carrier components are amorphous, above the softening point of one or more of the carrier components. The molten mixture is therefore often a suspension of solid particles in a fluid matrix. In one preferred embodiment, the molten mixture comprises a mixture of substantially crystalline azithromycin particles suspended in a carrier that is substantially fluid. In such cases, a portion of the azithromycin may be dissolved in the fluid carrier and a portion of the carrier may remain solid.

Although the term “melt” generally refers specifically to the transition of a crystalline material from its crystalline to its liquid state, which occurs at its melting point, and the term “molten” generally refers to such a crystalline material in its fluid state, as used herein, the terms are used more broadly, referring in the case of “melt” to the heating of any material or mixture of materials sufficiently that it becomes fluid in the sense that it may be pumped or atomized in a manner similar to a crystalline material in the fluid state. Likewise “molten” refers to any material or mixture of materials that is in such a fluid state.

Virtually any process can be used to form the molten mixture. One method involves melting the carrier in a tank, adding the azithromycin to the molten carrier, and then mixing the mixture to ensure the azithromycin is uniformly distributed therein. Alternatively, both the azithromycin and carrier may be added to the tank and the mixture heated and mixed to form the molten mixture. When the carrier comprises more than one material, the molten mixture may be prepared using two tanks, melting a first carrier in one tank and a second in another. The azithromycin is added to one of these tanks and mixed as described above. In another method, a continuously stirred tank system may be used, wherein the azithromycin and carrier are continuously added to a heated tank equipped with means for continuous mixing, while the molten mixture is continuously removed from the tank.

An especially preferred method of forming the molten mixture is by an extruder. By “extruder” is meant a device or collection of devices that creates a molten extrudate by heat and/or shear forces and/or produces a uniformly mixed extrudate from a solid and/or liquid (e.g., molten) feed. Such devices include, but are not limited to single-screw extruders; twin-screw extruders, including co-rotating, counter-rotating, intermeshing, and non-intermeshing extruders; multiple screw extruders; ram extruders, consisting of a heated cylinder and a piston for extruding the molten feed; gear-pump extruders, consisting of a heated gear pump, generally counter-rotating, that simultaneously heats and pumps the molten feed; and conveyer extruders. Conveyer extruders comprise a conveyer means for transporting solid and/or powdered feeds, such, such as a screw conveyer or pneumatic conveyer, and a pump. At least a portion of the conveyer means is heated to a sufficiently high temperature to produce the molten mixture. The molten mixture may optionally be directed to an accumulation tank, before being directed to a pump, which directs the molten mixture to an atomizer. Optionally, an in-line mixer may be used before or after the pump to ensure the molten mixture is substantially homogeneous. In each of these extruders the molten mixture is mixed to form a uniformly mixed extrudate. Such mixing may be accomplished by various mechanical and processing means, including mixing elements, kneading elements, and shear mixing by backflow. Thus, in such devices, the composition is fed to the extruder, which produces a molten mixture that can be directed to the atomizer.

The molten mixture may also be formed using a continuous mill, such as a Dyno® Mill wherein the azithromycin and carrier are typically fed in solid form to the mill's grinding chamber that contains grinding media, such as beads with diameters of 0.25 to 5 mm. The grinding chamber typically is jacketed so heating or cooling fluid may be circulated around the chamber to control its temperature. The molten mixture is formed in the grinding chamber, and exits the chamber through a separator to remove the grinding media from the molten mixture.

When preparing the molten mixture in which the composition contains azithromycin in a crystalline hydrate or solvate form, the azithromycin can be maintained in this form by ensuring that the activity of water or solvent in the molten mixture is sufficiently high such that the waters of hydration or solvate of the azithromycin crystals are not removed by dissolution into the molten mixture. To keep the activity of water or solvent in the molten mixture high, it is desirable to keep the gas phase atmosphere above the molten mixture at a high water or solvent activity. The inventors have found that when crystalline azithromycin dihydrate is contacted with a dry molten carrier and a dry gas-phase atmosphere, it can be converted into other less stable crystalline forms of azithromycin, such as the monohydrate. One method to ensure that crystalline azithromycin dihydrate is not converted to another crystalline form by virtue of loss of waters of hydration is to humidify the atmosphere in contact with the molten mixture during processing. Alternatively, a small amount of water, on the order of 30 to 100 wt % of the solubility of water in the molten carrier at the process temperature can be added to the molten mixture to ensure there is sufficient water to prevent loss of the azithromycin dihydrate crystalline form. This is disclosed more fully in commonly assigned U.S. patent application Ser. No. ______ (“Method for Making Pharmaceutical Multiparticulates,” Attorney Docket No. PC25021), filed concurrently herewith.

Once the molten mixture has been formed, it is delivered to an atomizer that breaks the molten feed into small droplets. Virtually any method can be used to deliver the molten mixture to the atomizer, including the use of pumps and various types of pneumatic devices such as pressurized vessels or piston pots. When an extruder is used to form the molten mixture, the extruder itself can be used to deliver the molten mixture to the atomizer. Typically, the molten mixture is maintained at an elevated temperature while delivering the mixture to the atomizer to prevent solidification of the mixture and to keep the molten mixture flowing.

The molten mixture is preferably molten prior to congealing for at least 5 seconds, more preferably for at least 10 seconds and most preferably for at least 15 seconds so as to ensure adequate homogeneity of the drug/carrier melt. The molten mixture preferably also remains molten for no more than about 20 minutes to limit formation of azithromycin esters. As described above, depending on the reactivity of the chosen carrier, it may be preferable to further reduce the time that the azithromycin mixture is molten to well below 20 minutes in order to further limit azithromycin ester formation to an acceptable level. In such cases, such mixtures may be maintained in the molten state for less than 15 minutes, and in some cases, even less than 10 minutes. When an extruder is used to produce the molten feed, the times above refer to the mean time from when material is introduced to the extruder to when the molten mixture is congealed. Such mean times can be determined by procedures well known in the art. In one exemplary method, a small amount of dye or other tracer substance is added to the feed while the extruder is operating under nominal conditions. Congealed multiparticulates are then collected over time and analyzed for the dye or tracer substance, from which the mean time is determined. In a particularly preferred embodiment the azithromycin is maintained substantially in the crystalline dihydrate state. To accomplish this, the feed is preferably hydrated by addition of water to a relative humidity of at least 30% at the maximum temperature of the molten mixture.

Generally, atomization occurs in one of several ways, including (1) by “pressure” or single-fluid nozzles; (2) by two-fluid nozzles; (3) by centrifugal or spinning-disk atomizers; (4) by ultrasonic nozzles; and (5) by mechanical vibrating nozzles. Detailed descriptions of atomization processes can be found in Lefebvre, Atomization and Sprays (1989) or in Perry's Chemical Engineers' Handbook, (7th Ed. 1997). In a preferred embodiment, the atomizer is a centrifugal or spinning-disk atomizer, such as the FX1 100-mm rotary atomizer manufactured by Niro A/S (Soeborg, Denmark).

Once the molten mixture has been atomized, the droplets are congealed, typically by contact with a gas or liquid at a temperature below the solidification temperature of the droplets. Typically, it is desirable that the droplets are congealed in less than about 60 seconds, preferably in less than about 10 seconds, more preferably in less than about 1 second. Often, congealing at ambient temperature results in sufficiently rapid solidification of the droplets to avoid excessive azithromycin ester formation. However, the congealing step often occurs in an enclosed space to simplify collection of the multiparticulates. In such cases, the temperature of the congealing media (either gas or liquid) will increase over time as the droplets are introduced into the enclosed space, leading to the possible formation of azithromycin esters. Thus, a cooling gas or liquid is often circulated through the enclosed space to maintain a constant congealing temperature. When the carrier used is highly reactive with azithromycin, the time the azithromycin is exposed to the molten carrier must be kept to an acceptably low level. In such cases, the cooling gas or liquid can be cooled to below ambient temperature to promote rapid congealing, thus further reducing the formation of azithromycin esters.

In another aspect, the multiparticulates are made by a liquid-based process comprising the steps of (a) forming a mixture comprising azithromycin, a pharmaceutically acceptable carrier, a pharmaceutically acceptable dissolution enhancer, and a liquid; (b) forming particles from the mixture of step (a); and (c) removing a substantial portion of the liquid from the particles of step (b) to form multiparticulates. Preferably, step (b) is a method selected from (i) atomization of the mixture (e.g., spray drying), (ii) coating seed cores with the mixture, (iii) wet-granulating the mixture and (iv) extruding the mixture into a solid mass followed by spheronizing or milling the mass.

Preferably, the liquid has a boiling point of less than about 150° C. Examples of liquids suitable for formation of multiparticulates using liquid-based processes include water; alcohols, such as methanol, ethanol, various isomers of propanol and various isomers of butanol; ketones, such as acetone, methyl ethyl ketone and methyl isobutyl ketone; hydrocarbons, such as pentane, hexane, heptane, cyclohexane, methylcyclohexane, octane and mineral oil; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; N,N-dimethylacetamide; acetonitrile; and mixtures thereof.

In one embodiment, the particles are formed by atomization of the mixture using an appropriate nozzle to form small droplets of the mixture, which are sprayed into a drying chamber where there is a strong driving force for evaporation of the liquid, to produce solid, generally spherical particles. The strong driving force for evaporation of the liquid is generally provided by maintaining the partial pressure of liquid in the drying chamber well below the vapor pressure of the liquid at the temperature of the particles. This is accomplished by (1) maintaining the pressure in the drying chamber at a partial vacuum (e.g., 0.01 to 0.5 atm); or (2) mixing the droplets with a warm drying gas; or (3) both (1) and (2). Spray-drying processes and spray-drying equipment are described generally in Perry's Chemical Engineers' Handbook, pages 20-54 to 20-57 (6th Ed. 1984).

In another embodiment, the particles are formed by coating the liquid mixture onto seed cores. The seed cores can be made from any suitable material such as starch, microcrystalline cellulose, sugar or wax, by any known method, such as melt- or spray-congealing, extrusion/spheronization, granulation, spray-drying and the like.

The liquid mixture can be sprayed onto such seed cores using coating equipment known in the pharmaceutical arts, such as pan coaters (e.g., Hi-Coater available from Freund Corp. of Tokyo, Japan, Accela-Cota available from Manesty of Liverpool, U.K.), fluidized bed coaters (e.g., Würster coaters or top-spray coaters, available from Glatt Air Technologies, Inc. of Ramsey, N.J. and from Niro Pharma Systems of Bubendorf, Switzerland) and rotary granulators (e.g., CF-Granulator, available from Freund Corp).

In another embodiment, the liquid mixture may be wet-granulated to form the particles. Granulation is a process by which relatively small particles are built up into larger granular particles, often with the aid of a carrier, also known as a binder in the pharmaceutical arts. In wet granulation, a liquid is used to increase the intermolecular forces between particles, leading to an enhancement in granular integrity, referred to as the “strength” of the granule. Often, the strength of the granule is determined by the amount of liquid that is present in the interstitial spaces between the particles during the granulation process. This being the case, it is important that the liquid wet the particles, ideally with a contact angle of zero. Since a large percentage of the particles being granulated are very hydrophilic azithromycin crystals, the liquid needs to be fairly hydrophilic to meet this criterion. Thus, effective wet granulation liquids tend also to be hydrophilic. Examples of liquids found to be effective wet granulation liquids include water, ethanol, isopropyl alcohol and acetone. Preferably, the wet granulation liquid is water at pH 7 or higher.

Several types of wet granulation processes can be used to form azithromycin-containing multiparticulates. Examples include fluidized bed granulation, rotary granulation and high-shear mixers. In fluidized bed granulation, air is used to agitate or “fluidize” particles of azithromycin and/or carrier in a fluidizing chamber. The liquid is then sprayed into this fluidized bed, forming the granules. In rotary granulation, horizontal discs rotate at high speed, forming a rotating “rope” of azithromycin and/or carrier particles at the walls of the granulation vessel. The liquid is sprayed into this rope, forming the granules. High-shear mixers contain an agitator or impeller to mix the particles of azithromycin and/or carrier. The liquid is sprayed into the moving bed of particles, forming granules. In these processes, all or a portion of the carrier can be dissolved into the liquid prior to spraying the liquid onto the particles. Thus, in these processes, the steps of forming the liquid mixture and forming particles from the liquid mixture occur simultaneously.

In another embodiment, the particles are formed by extruding the liquid mixture into a solid mass followed by spheronizing or milling the mass. In this process, the liquid mixture, which is in the form of a paste-like plastic suspension, is extruded through a perforated plate or die to form a solid mass, often in the form of elongated, solid rods. This solid mass is then milled to form the multiparticulates. In one embodiment, the solid mass is placed, with or without an intervening drying step, onto a rotating disk that has protrusions that break the material into multiparticulate spheres, spheroids, or rounded rods. The so-formed multiparticulates are then dried to remove any remaining liquid. This process is sometimes referred to in the pharmaceutical arts as an extrusion/spheronization process.

Once the particles are formed, a portion of the liquid is removed, typically in a drying step, thus forming the multiparticulates. Preferably, at least 80% of the liquid is removed from the particles, more preferably at least 90%, and most preferably at least 95% of the liquid is removed from the particle during the drying step.

The multiparticulates may also be made by a granulation process comprising the steps of (a) forming a solid mixture comprising azithromycin and a pharmaceutically acceptable carrier; and (b) granulating said mixture to form multiparticulates. Examples of such granulation processes include dry granulation and melt granulation, well known in the art (see, for example, Remington's Pharmaceutical Sciences (18^{th} Ed. 1990).

An example of a dry granulation process is roller compaction. In roller compaction processes, the solid mixture is compressed between rollers. The rollers can be designed such that the resulting compressed material is in the form of small beads or pellets of the desired diameter. Alternatively, the compressed material is in the form of a ribbon that may be milled to for multiparticulates using methods well known in the arts. See, for example, Remington's Pharmaceutical Sciences (16th Ed. 1980).

In melt granulation processes, the solid mixture is fed to granulator that has the capability of heating or melting the carrier. Equipment suitable for use in this process includes high-shear granulators and single or multiple screw extruders such as those described above for melt-congeal processes. In melt granulation processes, the solid mixture is placed into the granulator and heated until the solid mixture agglomerates. The solid mixture is then kneaded or mixed until the desired particle size is attained. The so-formed granules are then cooled, removed from the granulator and sieved to the desired size fraction, thus forming the multiparticulates.

### Controlled Release

Multiparticulate compositions of the present invention are designed for controlled release of azithromycin after introduction to a use environment. By “controlled release” is meant sustained release, delayed release, and sustained release with a lag time. The composition can operate by effecting the release of azithromycin at a rate sufficiently slow to ameliorate side effects. The composition can also release the bulk of the azithromycin in the portion of the GI tract distal to the duodenum. In the following, reference to “azithromycin” in terms of therapeutic amounts or in release rates is to active azithromycin, i.e., the non-salt, non-hydrated macrolide molecule having a molecular weight of 749 g/mol.

In one aspect, the compositions formed by the inventive process release azithromycin according to the release profiles set forth in commonly assigned U.S. Pat. No. 6,068,859.

In another aspect, the compositions formed by the inventive process, following administration to a stirred buffered test medium comprising 900 mL of pH 6.0 Na₂HPO₄ buffer at 37° C., release azithromycin to said test medium at the following rate: (i) from about 15 to about 55 wt %, but no more than 1.1 gA of the azithromycin in the dosage form at 0.25 hour; (ii) from about 30 to about 75 wt %, but no more than 1.5 gA, preferably no more than 1.3 gA of the azithromycin in the dosage form at 0.5 hour; and (iii) greater than about 50 wt % of the azithromycin in the dosage form at 1 hour following administration to the buffered test medium. In addition, dosage forms containing the inventive compositions exhibit an azithromycin release profile for patient in a fasted state that achieves a maximum azithromycin blood concentration of at least 0.5 μg/mL in at least 2 hours from dosing and an area under the azithromycin blood concentration versus time curve of at least 10 μg.hr/mL within 96 hours of dosing.

The multiparticulates of the present invention may be mixed or blended with one or more pharmaceutically acceptable materials to form a suitable dosage form. Suitable dosage forms include tablets, capsules, sachets, oral powders for constitution and the like.

The multiparticulates may also be dosed with alkalizing agents to reduce the incidence of side effects. The term “alkalizing agents”, as used herein, means one or more pharmaceutically acceptable excipients that will raise the pH in a constituted suspension or in a patient's stomach after being orally administered to said patient. Alkalizing agents include, for example, antacids as well as other pharmaceutically acceptable (1) organic and inorganic bases, (2) salts of strong organic and inorganic acids, (3) salts of weak organic and inorganic acids, and (4) buffers. Exemplary alkalizing agents include, but are not limited to, aluminum salts such as magnesium aluminum silicate; magnesium salts such as magnesium carbonate, magnesium trisilicate, magnesium aluminum silicate, magnesium stearate; calcium salts such as calcium carbonate; bicarbonates such as calcium bicarbonate and sodium bicarbonate; phosphates such as monobasic calcium phosphate, dibasic calcium phosphate, dibasic sodium phosphate, tribasic sodium phosphate (TSP), dibasic potassium phosphate, tribasic potassium phosphate; metal hydroxides such as aluminum hydroxide, sodium hydroxide and magnesium hydroxide; metal oxides such as magnesium oxide; N-methyl glucamine; arginine and salts thereof; amines such as monoethanolamine, diethanolamine, triethanolamine, and tris(hydroxymethyl)aminomethane (TRIS); and combinations thereof. Preferably, the alkalizing agent is TRIS, magnesium hydroxide, magnesium oxide, dibasic sodium phosphate, TSP, dibasic potassium phosphate, tribasic potassium phosphate or a combination thereof. More preferably, the alkalizing agent is a combination of TSP and magnesium hydroxide. Alkalizing agents are disclosed more fully for azithromycin-containing multiparticulates in commonly assigned U.S. patent application Ser. No. ______ (“Azithromycin Dosage Forms With Reduced Side Effects,” Attorney Docket No. PC25240), filed concurrently herewith.

The multiparticulates of the present invention may be post-treated to improve drug crystallinity and/or the stability of the multiparticulate. In one embodiment, the multiparticulates comprise azithromycin and a carrier, the carrier having a melting point of Tₘ° C.; the multiparticulates are treated after formation by at least one of (i) heating the multiparticulates to a temperature of at least about 35° C. and less than about (Tₘ° C.-10° C.), and (ii) exposing the multiparticulates to a mobility-enhancing agent. Such a post-treatment step results in an increase in drug crystallinity in the multiparticulates, and typically in an improvement in at least one of the chemical stability, physical stability, and dissolution stability of the multiparticulates. Post-treatment processes are disclosed more fully in commonly assigned U.S. patent application Ser. No. ______, (“Multiparticulate Compositions with Improved Stability,” Attorney Docket No. PC11900) filed concurrently herewith.

Without further elaboration, it is believed that one of ordinary skill in the art can, using the foregoing description, utilize the present invention to its fullest extent. Therefore, the following specific embodiments are to be construed as merely illustrative and not restrictive of the scope of the invention. Those of ordinary skill in the art will understand that known variations of the conditions and processes of the following examples can be used.

### SCREENING EXAMPLES 1-3

The tendency of azithromycin to form esters in melts at different temperatures and for different periods of time was studied. A mixture of glyceryl behenates (13 to 21 wt % monobehenate, 40 to 60 wt % dibehenate, and 21 to 35 wt % tribehenate)(COMPRITOL 888 ATO from Gattefossé Corporation of Paramus, N.J.), was deposited in 2.5 g samples into glass vials and melted in a temperature-controlled oil bath at 100° C. (Example 1), 90° C. (Example 2), and 80° C. (Example 3). To each of these three melts was then added 2.5 g of azithromycin dihydrate, thereby forming a suspension of the azithromycin in the molten COMPRITOL 888 ATO. After stirring the suspension for 15 minutes, a 50 to 100 mg sample of the suspension was removed from each of the molten samples and congealed by allowing the same to cool to room temperature. With stirring of each suspension continuing, additional samples were collected at the elapse of 30, 60, and 120 minutes following formation of the suspension. All collected samples were stored at −20° C. until analyzed.

Azithromycin esters were identified in each sample by Liquid Chromatography/Mass Spectrometer (LC/MS) Analysis using a Finnegan LCQ Classic mass spectrometer. Samples having a 1.25 mg/mL concentration of azithromycin were prepared by extraction with isopropyl alcohol and sonicated for 15 minutes. The samples were then filtered with a 0.45 μm nylon syringe filter, then analyzed by High Performance Liquid Chromatography (HPLC) using a Hypersil BDS C18 4.6 mm×250 mm (5 μm) HPLC column on a Hewlett Packard HP1100 liquid chromatograph. The mobile phase employed for sample elution was a gradient of isopropyl alcohol and 25 mM ammonium acetate buffer (pH approximately 7) of the following composition: initial conditions of 50/50 (v/v) isopropyl alcohol/ammonium acetate; the isopropyl alcohol percentage was then increased to 100% over 30 minutes and held at 100% for an additional 15 minutes. The flow rate was 0.80 mL/min. A 75 μL injection volume and a 43° C. column temperature were used.

LC/MS was used for detection with an Atmospheric Pressure Chemical Ionization (APCI) source used in positive-ion mode with selective ion-monitoring. Azithromycin ester formation was calculated from the mass spectrometer peak areas based on an azithromycin control. The azithromycin ester values are reported as percentages of the total azithromycin in the sample. The results of the tests are shown in Table 1, and indicate that the longer the azithromycin was in the molten suspension, and the higher the melt temperature, the greater was the concentration of azithromycin esters.

TABLE 1

| Screening | Melt | Exposure Time | Ester Concentration |
|---|---|---|---|
| Example | Temperature | (days) | (wt %) |
| 1 | 100° C. | 0 | 0.00 |
| | | 0.01 | 0.13 |
| | | 0.02 | 0.34 |
| | | 0.04 | 0.38 |
| | | 0.08 | 0.92 |
| 2 | 90° C. | 0 | 0.00 |
| | | 0.01 | 0.09 |
| | | 0.02 | 0.19 |
| | | 0.04 | 0.35 |
| | | 0.08 | 0.49 |
| 3 | 80° C. | 0 | 0.00 |
| | | 0.01 | 0.05 |
| | | 0.02 | 0.13 |
| | | 0.04 | 0.15 |
| | | 0.08 | 0.38 |

These data were then fitted to Equation I above to describe the rate of azithromycin ester formation Rₑ in wt %/day:
Rₑ=Cₑₛₜₑᵣₛ÷t.

The reaction rates calculated from the data in Table 1 are reported in Table 2.

TABLE 2

| Screening | Melt | Rₑ |
|---|---|---|
| Example | Temperature | wt %/day) |
| 1 | 100° C. | 10.4 |
| 2 | 90° C. | 5.8 |
| 3 | 80° C. | 4.4 |

### SCREENING EXAMPLES 4-25

The tendency of azithromycin to form esters in melts at different temperatures and for different periods of time was studied. Screening Examples 4-25 were prepared like Examples 1-3 except that a variety of different carriers, dissolution enhancers, temperatures, and exposure times were used, all as tabulated in Table 3. The chemical makeup of the various carriers screened is as follows: MYVAPLEX 600 is a glyceryl monostearate; GELUCIRE 50/13 is a mixture of mono-, di- and tri-alkyl glycerides and mono- and di-fatty acid esters of polyethylene glycol; carnauba wax is a complex mixture of esters of acids and hydroxyacids, oxypolyhydric alcohols, hydrocarbons, resinous matter, and water; microcrystalline wax is a petroleum-derived mixture of straight chain and randomly branched saturated alkanes obtained from petroleum; paraffin wax is a purified mixture of solid saturated hydrocarbons; stearyl alcohol is 1-octadecanol; stearic acid is octadecanoic acid; PLURONIC F127 is a block copolymer of ethylene oxide and propylene oxide, referred to as poloxamer 407, and also sold as LUTROL F127; PEG 8000 is a polyethylene glycol having a molecular weight of 8000 daltons; BRIJ 76 is a polyoxyl 10 stearyl ether; MYRJ 59 is a polyoxyethylene stearate; TWEEN 80 is a polyoxyethylene 20 sorbitan monooleate. Table 3 also reports the concentration of azithromycin esters formed. Table 4 reports the calculated reaction rates.

TABLE 3

| | | Melt | | Esters |
|---|---|---|---|---|
| Screening | | Temperature | Exposure | Formed |
| Example | Excipient | (° C.) | (day) | (wt %) |
| 4 | MYVAPLEX | 100 | 0 | 0 |
| | 600 | | 0.01 | 0.60 |
| | | | 0.02 | 1.14 |
| | | | 0.04 | 1.90 |
| | | | 0.08 | 3.28 |
| 5 | MYVAPLEX | 90 | 0 | 0 |
| | 600 | | 0.01 | 0.37 |
| | | | 0.02 | 0.87 |
| | | | 0.04 | 1.33 |
| | | | 0.08 | 1.93 |
| 6 | MYVAPLEX | 80 | 0 | 0 |
| | 600 | | 0.01 | 0.26 |
| | | | 0.02 | 0.55 |
| | | | 0.04 | 0.92 |
| | | | 0.08 | 1.71 |
| 7 | GELUCIRE | 80 | 0 | 0 |
| | 50/13 | | 0.04 | 0.035 |
| | | | 0.08 | 0.049 |
| 8 | GELUCIRE | 100 | 0 | 0 |
| | 50/13 | | 0.04 | 0.084 |
| | | | 0.08 | 0.134 |
| 9 | carnauba wax | 90 | 0 | 0 |
| | | | 0.04 | 0.012 |
| | | | 0.08 | 0.015 |
| 10 | carnauba wax | 100 | 0 | 0 |
| | | | 0.04 | 0.012 |
| | | | 0.08 | 0.015 |
| 11 | microcrystalline | 100 | 0 | 0 |
| | wax | | 0.08 | 0.002 |
| 12 | paraffin wax | 100 | 0 | 0 |
| | | | 0.08 | 0.000 |
| 13 | stearyl alcohol | 80 | 0 | 0 |
| | | | 0.04 | 0.0001 |
| | | | 0.08 | 0.0003 |
| 14 | stearyl alcohol | 100 | 0 | 0 |
| | | | 0.04 | 0.0002 |
| | | | 0.08 | 0.0001 |
| 15 | stearic acid | 80 | 0 | 0 |
| | | | 0.04 | 0.704 |
| | | | 0.08 | 1.718 |
| 16 | stearic acid | 100 | 0 | 0 |
| | | | 0.04 | 3.038 |
| | | | 0.08 | 5.614 |
| 18 | PLURONIC | 100 | 0 | 0 |
| | F127 | | 0.04 | 0.0005 |
| | | | 0.08 | 0.0001 |
| 19 | PEG 8000 | 100 | 0 | 0 |
| | | | 0.04 | 0 |
| | | | 0.08 | 0 |
| 20 | BRIJ 76 | 80 | 0 | 0 |
| | | | 0.04 | 0.0014 |
| | | | 0.08 | 0.0015 |
| 21 | BRIJ 76 | 100 | 0 | 0 |
| | | | 0.04 | 0.0013 |
| | | | 0.08 | 0.0081 |
| 22 | MYRJ 59 | 80 | 0 | 0 |
| | | | 0.04 | 0.0017 |
| | | | 0.08 | 0.0023 |
| 23 | MYRJ 59 | 100 | 0 | 0 |
| | | | 0.04 | 0.0027 |
| | | | 0.08 | 0.0042 |
| 24 | TWEEN 80 | 80 | 0 | 0 |
| | | | 0.04 | 0.0035 |
| | | | 0.08 | 0.0136 |
| 25 | TWEEN 80 | 100 | 0 | 0 |
| | | | 0.04 | 0.0193 |
| | | | 0.08 | 0.0221 |

TABLE 4

| Screening | | Melt Temp. | Rₑ |
|---|---|---|---|
| Example | Excipient | (° C.) | (wt %/day) |
| 4 | MYVAPLEX 600 | 100 | 38.0 |
| 5 | MYVAPLEX 600 | 90 | 22.5 |
| 6 | MYVAPLEX 600 | 80 | 19.9 |
| 7 | GELUCIRE 50/13 | 80 | 0.059 |
| 8 | GELUCIRE 50/13 | 100 | 1.64 |
| 9 | carnauba wax | 90 | 0.18 |
| 10 | carnauba wax | 100 | 0.23 |
| 11 | microcrystalline wax | 100 | 0 |
| 12 | paraffin wax | 100 | 0 |
| 13 | stearyl alcohol | 80 | 0.0018 |
| 14 | stearyl alcohol | 100 | 0.0047 |
| 15 | stearic acid | 80 | 20.7 |
| 16 | stearic acid | 100 | 67.4 |
| 17 | PLURONIC F127 | 80 | 0.0005 |
| 18 | PLURONIC F127 | 100 | 0.001 |
| 19 | PEG 8000 | 100 | 0 |
| 20 | BRIJ 76 | 80 | 0.018 |
| 21 | BRIJ 76 | 100 | 0.095 |
| 22 | MYRJ 59 | 80 | 0.029 |
| 23 | MYRJ 59 | 100 | 0.051 |
| 24 | TWEEN 80 | 80 | 0.16 |
| 25 | TWEEN 80 | 100 | 0.27 |

The high reaction rates for MYVAPLEX 600 and stearic acid indicate that these carriers are not suitable candidates.

### SCREENING EXAMPLE 26

This example illustrates how the degree of acid/ester substitution can be determined from the Saponification Number for an excipient. The degree of acid/ester substitution [A] for the candidate excipients listed in Table 5 was determined by dividing by 56.11 the Saponification Number for the excipient as listed in Pharmaceutical Excipients 2000.

TABLE 5

| | Saponification | |
|---|---|---|
| Excipient | Number | [A]* |
| hydrogenated castor oil | 176-182 | 3.1-3.2 |
| cetostearyl alcohol | <2 | <0.04 |
| cetyl alcohol | <2 | <0.04 |
| glyceryl monooleate | 160-170 | 2.9-3.0 |
| glyceryl monostearate | 155-165 | 2.8-2.9 |
| glyceryl palmitostearate | 175-195 | 3.1-3.5 |
| lecithin | 196 | 3.5 |
| polyoxyethylene alkyl ether | <2 | <0.04 |
| polyoxyethylene castor oil derivatives | 40-50 | 0.7-0.9 |
| polyoxyethylene sorbitan fatty acid | 45-55 | 0.8-1.0 |
| esters | | |
| polyoxyethylene stearates | 25-35 | 0.4-0.6 |
| sorbitan monostearate | 147-157 | 2.6-2.8 |
| stearic acid | 200-220 | 3.6-3.9 |
| stearyl alcohol | <2 | <0.04 |
| anionic emulsifying wax | <2 | <0.04 |
| carnauba wax | 78-95 | 1.4-1.7 |
| cetyl esters wax | 109-120 | 1.9-2.1 |
| microcrystalline wax | 0.05-0.1 | 0.001-0.002 |
| nonionic emulsifying wax | <14 | <0.25 |
| white wax | 87-104 | 1.6-1.9 |
| yellow wax | 87-102 | 1.6-1.8 |

*meq/g carrier

### SCREENING EXAMPLE 27

This example illustrates how the degree of acid/ester substitution can be determined from the Saponification Number for an excipient. The degree of acid/ester substitution for the candidate carriers and excipients listed in Table 6 were determined by dividing by 56.11 the Saponification Number provided by the manufacturer.

TABLE 6

| | Saponification | |
|---|---|---|
| Excipient | Number | [A]* |
| COMPRITOL 888 ATO | 145-165 | 2.6-2.9 |
| GELUCIRE 50/13 | 67-81 | 1.2-1.4 |

*meq/g carrier

### SCREENING EXAMPLE 28

This example illustrates how the degree of acid/ester substitution can be determined from the structure of the excipient. The degree of acid/ester substitution for the candidate carriers and excipients listed in Table 7 was determined by dividing the number of moles of acid and ester substituents on the carrier by its molecular weight. For polymers, the degree of acid/ester substitution was calculated by dividing the average number of moles of acid and ester substituents on the monomer by the monomer's molecular weight.

TABLE 7

| | Molecular | Acid and Ester | |
|---|---|---|---|
| | Weight | Substituents | |
| Excipient | (g/mol) | per mol | [A]* |
| PLURONIC F127 | 10,000 | 0 | 0 |
| paraffin wax | 500 | 0 | 0 |
| PEG 8000 | 8,000 | 0 | 0 |
| triacetin | 218 | 3 | 14 |

*meq/g carrier

### SCREENING EXAMPLE 29

The solubility of azithromycin dihydrate in beeswax was measured using the following procedure. A 5 g sample of beeswax was placed in a glass vial and melted at 65° C. by placing the vial in a hot-water bath. Crystals of azithromycin dihydrate were then slowly added to the molten wax, with stirring. The crystals first added dissolved into the wax. When a total of 0.3 g azithromycin dihydrate had been added to the molten wax, all of the azithromycin dihydrate dissolved into the wax, whereas when an additional 0.1 g of azithromycin dihydrate was added, the crystals did not dissolve after stirring for 30 minutes. Thus, the solubility of azithromycin dihydrate in beeswax was determined to be about 6 wt %.

### SCREENING EXAMPLES 30-40

Using the procedure outlined in Screening Example 29, the solubility of azithromycin dihydrate in the carriers and excipients listed in Table 8 was determined at the temperatures listed therein. In addition, the solubility of azithromycin dihydrate was determined for mixtures of carriers in the weight ratios reported in Table 8.

TABLE 8

| | | | Azithromycin |
|---|---|---|---|
| Screening | | Temperature | Solubility |
| Example | Excipient | (° C.) | (wt %) |
| 30 | carnauba wax | 95 | 6 |
| 31 | COMPRITOL 888 ATO | 85 | 6 |
| | (glyceryl behenate) | | |
| 32 | paraffin wax | 75 | 5 |
| 33 | MYVAPLEX 600P (glyceryl | 90 | >75 |
| | monostearate) | | |
| 34 | GELUCIRE 50/13 | 90 | 67 |
| 35 | MYRJ 59 (polyoxyethylene | 90 | <1 |
| | stearate) | | |
| 36 | BRIJ 76 (polyoxyethylene | 90 | 1 |
| | alkyl ether) | | |
| 37 | stearyl alcohol | 95 | 60 |
| 38 | 4:1 COMPRITOL 888 | 100 | 25 |
| | ATO:PLURONIC F127 | | |
| 39 | 4:1 carnauba | 90 | 13 |
| | wax:PLURONIC F127 | | |
| 40 | 4:1 COMPRITOL 888 | 85 | 7.5 |
| | ATO:GELUCIRE 51/13 | | |

### EXAMPLE 1

This example illustrates forming multiparticulates of the present invention by extruding a molten mixture to an atomizer and congealing the resulting droplets. Multiparticulates were prepared comprising 50 wt % azithromycin dihydrate, 45 wt % COMPRITOL 888 ATO as a carrier, and 5 wt % PLURONIC F127 as a dissolution enhancer. The concentration of acid and ester substituents on the dissolution enhancer was essentially 0 meq/g azithromycin. The multiparticulates were prepared using the following melt-congeal procedure. First, 112.5 g of the COMPRITOL 888 ATO, 12.5 g of the PLURONIC F127, and 2 g of water were added to a sealed, jacketed stainless-steel tank equipped with a mechanical mixing paddle. Heating fluid at 97° C. was circulated through the jacket of the tank. After about 40 minutes, the mixture had melted, having a temperature of about 95° C. This mixture was then mixed at 370 rpm for 15 minutes. Next, 125 g of at least 70% azithromycin crystalline dihydrate that had been pre-heated at 95° C. and 100% RH was added to the melt and mixed at a speed of 370 rpm for 5 minutes, resulting in a feed suspension of the azithromycin dihydrate in the molten components.

Using a gear pump, the feed suspension was then pumped at a rate of 250 g/min to the center of a spinning-disk atomizer. The spinning disk atomizer, which was custom made, consists of a bowl-shaped stainless steel disk of 10.1 cm (4 inches) in diameter. The surface of the disk is heated with a thin film heater beneath the disk to about 90° C. That disk is mounted on a motor that drives the disk of up to approximately 10,000 RPM. The entire assembly is enclosed in a plastic bag of approximately 8 feet in diameter to allow congealing and to capture microparticulates formed by the atomizer. Air is introduced from a port underneath the disk to provide cooling of the multiparticulates upon congealing and to inflate the bag to its extended size and shape.

A suitable commercial equivalent, to this spinning disk atomizer, is the FX1 100-mm rotary atomizer manufactured by Niro A/S (Soeborg, Denmark).

The surface of the spinning disk atomizer was maintained at 100° C., and the disk was rotated at 7500 rpm, while forming the azithromycin multiparticulates. The particles formed by the spinning-disk atomizer were congealed in ambient air and a total of 205 g of multiparticulates collected. The mean particle size was determined to be 170 μm using a Horiba LA-910 particle size analyzer. Samples of the multiparticulates were also evaluated by PXRD, which showed that 83±10% of the azithromycin in the multiparticulates was crystalline dihydrate.

The rate of release of azithromycin from these multiparticulates was determined using the following procedure. A 750 mg sample of the multiparticulates was placed into a USP Type 2 dissoette flask equipped with Teflon-coated paddles rotating at 50 rpm. The flask contained 750 mL of 0.01 N HCl (pH 2) simulated gastric buffer held at 37.0±0.5° C. The multiparticulates were pre-wet with 10 mL of the simulated gastric buffer before being added to the flask. A 3-mL sample of the fluid in the flask was then collected at 5, 15, 30, and 60 minutes following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer).

The results of this dissolution test are reported in Table 9, and confirm that controlled release of azithromycin from the multiparticulates was achieved.

TABLE 9

| | Azithromycin |
|---|---|
| Time (min) | Released (%) |
| 0 | 0 |
| 5 | 7.5 |
| 15 | 24.6 |
| 30 | 44.7 |
| 60 | 73.0 |

Samples of the multiparticulates were analyzed for azithromycin esters by LC/MS as in Screening Examples 1-3. The results of this analysis showed that the concentration of azithromycin esters in the multiparticulates was 0.05 wt %.

### EXAMPLES 2-3

Multiparticulates were prepared using the following melt-congeal procedure. For Example 2, the multiparticulates comprised 50 wt % azithromycin, at least 70% of which was in the crystalline dihydrate form; 45 wt % COMPRITOL 888 ATO as carrier; and 5 wt % PLURONIC F127 as dissolution enhancer. For Example 3, the multiparticulates comprised 50 wt % of the same azithromycin dihydrate, 46 wt % COMPRITOL 888 ATO, and 4 wt % PLURONIC F127. Thus, the concentration of acid and ester substituents on the dissolution enhancer for both Examples 2 and 3 was essentially 0 meq/g azithromycin. For Example 2, a mixture of 2.5 kg azithromycin dihydrate, 2.25 kg COMPRITOL 888 ATO, and 0.25 kg of PLURONIC F127 was blended in a V-blender for 20 minutes. This blend was then de-lumped using a Fitzpatrick M5A mill at 1000 rpm, knives forward using a 0.0065-inch screen, forming a preblend feed. For Example 3, 2.5 kg azithromycin dihydrate, 2.3 kg COMPRITOL 888 ATO, and 0.2 kg PLURONIC F127 were blended in a V-blender for 20 minutes. This blend was then de-lumped using a Fitzpatrick M5A mill at 1000 rpm, knives forward using a 0.0065-inch screen, forming a preblend feed.

The preblend feed was delivered to a B&P 19 mm twin-screw extruder at a rate of 115 g/min for Example 2 and at a rate of 120 g/min for Example 3. The extruder's rate of extrusion was set such that it produced a molten feed suspension of the azithromycin dihydrate in COMPRITOL 888 ATO/PLURONIC F127 at a temperature of about 90° C. The feed suspension was then delivered to the spinning-disk atomizer of Example 1, maintained at 90° C. and rotating at about 5500 rpm. The maximum residence time of azithromycin in the twin-screw extruder was about 60 seconds, and the total time the azithromycin was exposed to the molten suspension was less than about 3 minutes.

For Example 2, the resulting multiparticulates had a mean particle size of 190 μm and 80±4% of the azithromycin in the multiparticulates was crystalline dihydrate. For Example 3, the resulting multiparticulates had a mean particle size of 200 μm and 77±11% of the azithromycin in the multiparticulates was crystalline dihydrate.

The rate of azithromycin release from the multiparticulates was measured as in Example 1. The results are reported in Table 10.

TABLE 10

| | | Azithromycin |
|---|---|---|
| Example | Time (min) | Released (%) |
| 2 | 0 | 0 |
| | 5 | 4.9 |
| | 15 | 13.9 |
| | 30 | 28.1 |
| | 60 | 50.4 |
| 3 | 0 | 0 |
| | 5 | 3.2 |
| | 15 | 8.6 |
| | 30 | 18.7 |
| | 60 | 33.7 |

Samples of the multiparticulates were analyzed for azithromycin esters by LC/MS as in Screening Examples 1-3. The results of this analysis showed that the concentration of azithromycin esters in the multiparticulates of Example 2 was 0.01 wt %, while the concentration of azithromycin esters in the multiparticulates of Example 3 was 0.013%.

Samples of the multiparticulates were then stored under the accelerated aging conditions shown in Table 11. At the times indicated, samples were analyzed for azithromycin esters by LC/MS as in Screening Examples 1-3. As these data show, the concentration of azithromycin esters remained low in these samples.

TABLE 11

| | Storage | Storage | Concentration of | |
|---|---|---|---|---|
| Storage | Conditions | Time | Azithromycin Esters (wt %) | |
| Container | (° C./RH %) | (days) | Example 2 | Example 3 |
| Open | 40/75 | 5 | 0.028 | 0.033 |
| Open | 40/75 | 19 | 0.040 | Not determined |
| Foil/Foil | 40/75 | 21 | 0.039 | 0.047 |
| Amber Bottle | 40/75 | 21 | 0.036 | 0.048 |

### EXAMPLE 4

Multiparticulates were prepared comprising 50 wt % azithromycin dihydrate, 45 wt % carnauba wax as a carrier, and 5 wt % PLURONIC F127 as a dissolution enhancer. Thus, the concentration of acid and ester substituents on the carrier was about 1.5 meq/g, while that for the dissolution enhancer was essentially 0 meq/g azithromycin. The multiparticulates were prepared using the following melt-congeal procedure. First, 112.5 g of carnauba wax and 12.5 g of the PLURONIC F127 were melted in a vessel at a temperature of about 93° C. Next, 125 g of azithromycin at least 70% of which was in the crystalline dihydrate form was suspended in this melt and mixed by hand for about 15 minutes, resulting in a feed suspension of the azithromycin in the molten components.

Using a gear pump, the feed suspension was then pumped at a rate of 250 g/min to the center of the spinning-disk atomizer of Example 1, rotating at 5000 rpm, the surface of which was maintained at about 98° C. The particles formed by the spinning-disk atomizer were congealed in ambient air and a total of 167 g of multiparticulates collected.

The rate of release of azithromycin from these multiparticulates was determined as in Example 1. The results of this dissolution test are reported in Table 12, and show that controlled release of azithromycin from the multiparticulates was achieved.

TABLE 12

| | Azithromycin |
|---|---|
| Time (min) | Released (%) |
| 0 | 0 |
| 5 | 4 |
| 10 | 7 |
| 15 | 12 |
| 30 | 28 |
| 45 | 40 |
| 60 | 50 |

Samples of the multiparticulates were stored at room temperature for about 190 days and then analyzed for azithromycin esters by LC/MS as in Screening Examples 1-3. The results of this analysis showed that the concentration of azithromycin esters in the multiparticulates was 0.012 wt %.

### EXAMPLE 5

Multiparticulates were prepared comprising 38 wt % azithromycin dihydrate; 33 wt % microcrystalline wax as carrier; and 13 wt % Na₃PO₄, 8 wt % PLURONIC F87, and 8 wt % stearyl alcohol as dissolution enhancers. The concentration of acid and ester substituents on the carrier was about 0.002 meq/g, while that for the blended dissolution enhancer was less than 0.06 meq/g azithromycin. The multiparticulates were prepared using the following melt-congeal procedure. First, 166.5 g microcrystalline wax, 62.5 g Na₃PO₄, 41.5 g of the PLURONIC F87 and 41.5 g stearyl alcohol were heated in a glass beaker in a 95° C. water bath. After about 60 minutes, the mixture had melted. Next, 187.5 g of azithromycin at least 70% of which was in the crystalline dihydrate form was added to the melt and mixed using a spatula for about 15 minutes, resulting in a feed suspension of the azithromycin and the Na₃PO₄ in the other components.

Using a gear pump, the feed suspension was then pumped at a rate of 250 cc/min to the center of the spinning-disk atomizer of Example 1, rotating at 7000 rpm, the surface of which was maintained at about 100° C. The particles formed by the spinning-disk atomizer were congealed in ambient air. The mean particle size was determined to be 250 μm using a Horiba LA-910 particle-size analyzer. Samples of the multiparticulates were also evaluated by PXRD, which showed that about 89% of the azithromycin in the multiparticulates were crystalline dihydrate.

Samples of the multiparticulates were analyzed for azithromycin esters as in Screening Examples 1-3. No azithromycin esters were detected in these multiparticulates.

The rate of release of azithromycin from these multiparticulates was determined as in Example 1. The results of this dissolution test are reported in Table 13, and show that controlled release of azithromycin was achieved.

TABLE 13

| | Azithromycin |
|---|---|
| Time (min) | Released (%) |
| 0 | 0 |
| 5 | 38 |
| 10 | 61 |
| 15 | 78 |
| 30 | 90 |
| 45 | 95 |
| 60 | 97 |

### EXAMPLE 6

Multiparticulates were prepared comprising 45 wt % azithromycin dihydrate; 37 wt % microcrystalline wax as carrier; and 9 wt % PLURONIC F87 and 9 wt % stearyl alcohol as dissolution enhancers. The concentration of acid and ester substituents on the carrier and dissolution enhancer blend was substantially the same as for Example 5. The multiparticulates were prepared using the following melt-congeal procedure. First, 370 g microcrystalline wax, 90 g of the PLURONIC F87 and 90 g stearyl alcohol were heated in a glass beaker in a 93° C. water bath. After about 60 minutes, the mixture had melted. Next, 450 g of azithromycin dihydrate of the type used in Example 5 was added to the melt and mixed using a spatula for about 25 minutes, resulting in a feed suspension of the azithromycin in the other components.

Using a gear pump, the feed suspension was then pumped at a rate of 250 cc/min to the center of the spinning-disk atomizer of Example 1, rotating at 8000 rpm, the surface of which was maintained at about 100° C. The particles formed by the spinning-disk atomizer were congealed in ambient air. The mean particle size was determined to be 190 μm using a Horiba LA-910 particle-size analyzer. Samples of the multiparticulates were also evaluated by PXRD, which showed that about 84% of the azithromycin in the multiparticulates were crystalline dihydrate.

Samples of the multiparticulates were analyzed for azithromycin esters as in Screening Examples 1-3. No azithromycin esters were detected in these multiparticulates.

The rate of release of azithromycin from these multiparticulates was determined as in Example 1. The results of this dissolution test are reported in Table 14, and show that controlled release of azithromycin from the multiparticulates was achieved.

TABLE 14

| | Azithromycin |
|---|---|
| Tim (min) | Released (%) |
| 0 | 0 |
| 5 | 54 |
| 10 | 83 |
| 15 | 98 |
| 30 | 96 |
| 45 | 95 |
| 60 | 94 |

### EXAMPLES 7-12

Multiparticulates were made as in Example 2 comprising azithromycin dihydrate, COMPRITOL 888 ATO, and PLURONIC F127 in varying ratios with the variables noted in Table 15. In all cases, the concentration of acid and ester substituents on the dissolution enhancer blend was essentially zero. Following formation the multiparticulates were stored at the conditions shown in Table 15 in a sealed container.

TABLE 15

| | Formulation | | | | | |
|---|---|---|---|---|---|---|
| | (Azithromycin/ | | | | | Storage |
| | COMPRITOL/ | Feed | Disk | Disk | Batch | Conditions |
| Ex. | PLURONIC)* | Rate | Speed | Temp | Size | (° C./% RH; |
| No. | (wt %) | (g/min) | (rpm) | (° C.) | (g) | days) |
| 7 | 50/40/10 | 130 | 5500 | 90 | 500 | 47/70; 1 |
| 8 | 50/45/5 | 140 | 5500 | 90 | 491 | 47/70; 1 |
| 9 | 50/46/4 | 140 | 5500 | 90 | 4968 | 40/75; 5 |
| 10 | 50/47/3** | 180 | 5500 | 86 | 1015 | 40/75; 5 |
| 11 | 50/48/2 | 130 | 5500 | 90 | 500 | 47/70; 1 |
| 12 | 50/50/0 | 130 | 5500 | 90 | 500 | 47/70; 1 |

*COMPRITOL = COMPRITOL 888 ATO; PLURONIC = PLURONIC F127
**3.45 wt % water added to preblend feed.

The azithromycin release rate from the multiparticulates of Examples 7-12 was determined using the following procedure. A sample of the multiparticulates was placed into a USP Type 2 dissoette flask equipped with Teflon-coated paddles rotating at 50 rpm. For Examples 7-9 and 12, 1060 mg of multiparticulates were added to the dissolution medium; for Example 10, 1048 mg was added; for Example 11, 1000 mg was added. The flask contained 1000 mL of 50 mM KH₂PO₄ buffer, pH 6.8, maintained at 37.0±0.5° C. The multiparticulates were pre-wet with 10 mL of the buffer before being added to the flask. A 3-mL sample of the fluid in the flask was then collected at 5, 15, 30, 60, 120, and 180 minutes following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer). The results of these dissolution tests are reported in Table 16, and show that controlled release of azithromycin was achieved.

TABLE 16

| | | Azithromycin |
|---|---|---|
| Example No. | Time (min) | Released (%) |
| 7 | 0 | 0 |
| | 5 | 32 |
| | 15 | 67 |
| | 30 | 90 |
| | 60 | 99 |
| | 120 | 99 |
| | 180 | 100 |
| 8 | 0 | 0 |
| | 15 | 28 |
| | 30 | 46 |
| | 60 | 69 |
| | 120 | 87 |
| | 180 | 90 |
| 9 | 0 | 0 |
| | 15 | 25 |
| | 30 | 42 |
| | 60 | 64 |
| | 120 | 86 |
| | 180 | 93 |
| 10 | 0 | 0 |
| | 15 | 14 |
| | 30 | 27 |
| | 60 | 44 |
| | 120 | 68 |
| | 180 | 81 |
| 11 | 0 | 0 |
| | 5 | 3 |
| | 15 | 11 |
| | 30 | 23 |
| | 60 | 41 |
| | 120 | 66 |
| | 180 | 81 |
| 12 | 0 | 0 |
| | 5 | 4 |
| | 15 | 10 |
| | 30 | 19 |
| | 60 | 32 |
| | 120 | 50 |
| | 180 | 62 |

### EXAMPLE 13

Multiparticulates were made comprising 50 wt % azithromycin dihydrate, 47 wt % COMPRITOL 888 ATO, and 3 wt % PLURONIC F127 as dissolution enhancer. Thus, the concentration of acid and ester substituents on the dissolution enhancer was essentially zero. First, 15 kg azithromycin dihydrate, 14.1 kg of the COMPRITOL 888 ATO and 0.9 kg of the PLURONIC F127 were weighed and passed through a Quadro 194S Comil mill in the order listed above. The mill speed was set at 600 rpm. The mill was equipped with a No. 2C-075-H050/60 screen (special round), a No. 2C-1607-049 flat-blade impeller, and a 0.225-inch spacer between the impeller and screen. The milled mixture was blended using a Servo-Lift 100-L stainless-steel bin blender rotating at 20 rpm, for a total of 500 rotations, forming a preblend feed.

The preblend feed was delivered to a Leistritz 50 mm twin-screw extruder (Model ZSE 50, American Leistritz Extruder Corporation, Somerville, N.J.) at a rate of 25 kg/hr. The extruder was operated in co-rotating mode at about 300 rpm, and interfaced with a melt/spray-congeal unit. The extruder had nine segmented barrel zones and an overall extruder length of 36 screw diameters (1.8 m). Water was injected into barrel number 4 at a rate of 8.3 g/min. The extruder's rate of extrusion was set such that it produced a molten feed suspension of the azithromycin dihydrate in the COMPRITOL 888 ATO/PLURONIC F127 at a temperature of about 90° C.

The feed suspension was then delivered to the spinning-disk atomizer of Example 1, maintained at 90° C. and rotating at 7600 rpm. The maximum total time the azithromycin was exposed to the molten suspension was less than about 10 minutes. The particles formed by the spinning-disk atomizer were cooled and congealed in the presence of cooling air circulated through the product-collection chamber. The mean particle size was determined to be 188 μm using a Horiba LA-910 particle size analyzer. Samples of the multiparticulates were also evaluated by PXRD, which showed that about 99% of the azithromycin in the multiparticulates was in the crystalline dihydrate form.

The multiparticulates of Example 13 were post-treated by placing samples of the multiparticulates in sealed barrels, which were then placed in a controlled atmosphere chamber at 40° C. for 3 weeks.

The rate of release of azithromycin from the multiparticulates of Example 13 was determined using the following procedure. Approximately 4 g of the multiparticulates (containing about 2000 mgA of the drug) were placed into a 125 mL bottle containing approximately 21 g of a dosing vehicle consisting of the following excipients, all of which were NF grade with the exception of titanium dioxide: 92.3 wt % sucrose, 1.7 wt % trisodium phosphate, 1.2 wt % magnesium hydroxide, 0.3 wt % hydroxypropyl cellulose, 0.3 wt % xanthan gum, 0.5 wt % colloidal silicon dioxide, 1.9 wt % titanium dioxide (USP grade), 0.7 wt % cherry flavoring and 1.1 wt % banana flavoring. Next, 60 mL of purified water was added, and the bottle was shaken for 30 seconds. The contents were added to a USP Type 2 dissoette flask equipped with Teflon-coated paddles rotating at 50 rpm. The flask contained 840 mL of 100 mM Na₂HPO₄ buffer, pH 6.0, held at 37.0±0.5° C. The bottle was rinsed twice with 20 mL of the buffer from the flask, and the rinse was returned to the flask to make up a final volume of 900 mL. A 3-mL sample of the fluid in the flask was then collected at 15, 30, 60, 120, and 180 minutes following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer). The results of this dissolution test are reported in Table 17, and show that controlled release of the azithromycin was achieved.

TABLE 17

| | | Azithromycin |
|---|---|---|
| Example No. | Time (min) | Released (%) |
| 13 | 0 | 0 |
| | 15 | 28 |
| | 30 | 48 |
| | 60 | 74 |
| | 120 | 94 |
| | 180 | 98 |

### EXAMPLE 14

Multiparticulates were made comprising 50 wt % azithromycin dihydrate, 47 wt % COMPRITOL 888 ATO as carrier, and 3 wt % LUTROL F127 as dissolution enhancer. Thus, the concentration of acid and ester substituents on the dissolution enhancer was essentially zero. The following procedure was used. First, 140 kg azithromycin dihydrate was weighed and passed through a Quadro Comil 196S with a mill speed of 900 rpm. The mill was equipped with a No. 2C-075-H050/60 screen (special round, 0.075″), a No. 2F-1607-254 impeller, and a 0.225 inch spacer between the impeller and screen. Next, 8.4 kg of the LUTROL and then 131.6 kg of the COMPRITOL 888 ATO were weighed and passed through a Quadro 194S Comil mill. The mill speed was set at 650 rpm. The mill was equipped with a No. 2C-075-R03751 screen (0.075″), a No. 2C-1601-001 impeller, and a 0.225-inch spacer between the impeller and screen. The milled mixture was blended using a Gallay 38 cubic foot stainless-steel bin blender rotating at 10 rpm for 40 minutes, for a total of 400 rotations, forming a preblend feed.

The preblend feed was delivered to a Leistritz 50 mm twin-screw extruder (Model ZSE 50, American Leistritz Extruder Corporation, Somerville, N.J.) at a rate of about 20 kg/hr. The extruder was operated in co-rotating mode at about 100 rpm, and interfaced with a melt/spray-congeal unit. The extruder had five segmented barrel zones and an overall extruder length of 20 screw diameters (1.0 m). Water was injected into barrel number 2 at a rate of 6.7 g/min (2 wt %). The extruder's rate of extrusion was adjusted so as to produce a molten feed suspension of the azithromycin dihydrate in the COMPRITOL 888 ATO/LUTROL at a temperature of about 90° C.

The feed suspension was delivered to the spinning-disk atomizer of Example 1, rotating at 6400 rpm. The maximum total time the azithromycin was exposed to the molten suspension was less than 10 minutes. The particles formed by the spinning-disk atomizer were cooled and congealed in the presence of cooling air circulated through the product collection chamber. The mean particle size was determined to be about 200 μm using a Malvern particle size analyzer.

The so-formed multiparticulates were post-treated by placing a sample in a sealed barrel that was then placed in a controlled atmosphere chamber at 40° C. for 10 days. Samples of the post-treated multiparticulates were evaluated by PXRD, which showed that about 99% of the azithromycin in the multiparticulates was in the crystalline dihydrate form.

The rate of release of azithromycin from these multiparticulates was determined by placing a sample of the multiparticulates containing about 2000 mgA of azithromycin into a 125-mL bottle, along with the dosing excipients of Example 13. Next, 60 mL of purified water was added, and the bottle was shaken for 30 seconds. The contents were added to a USP Type 2 dissoette flask equipped with Teflon-coated paddles rotating at 50 rpm. The flask contained 840 mL of a buffered test solution comprising 100 mM Na₂HPO₄ buffer, pH 6.0, maintained at 37.0±0.5° C. The bottle was rinsed twice with 20 mL of the buffer from the flask, and the rinse was returned to the flask to make up a 900 mL final volume. A 3 mL sample of the fluid in the flask was then collected at 15, 30, 60, 120, and 180 minutes following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer). The results of these dissolution tests are given in Table 18, and show that sustained release of azithromycin was achieved.

TABLE 18

| | | Time | Azithromycin | Azithromycin |
|---|---|---|---|---|
| Example | Test Media | (min) | Released (mg) | Released (%) |
| 21 | 100 mM | 0 | 0 | 0 |
| | Na₂HPO₄ | 15 | 720 | 36 |
| | buffer, pH 6.0, | 30 | 1140 | 57 |
| | | 60 | 1620 | 81 |
| | | 120 | 1900 | 95 |
| | | 180 | 1960 | 98 |

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A pharmaceutical composition comprising multiparticulates, said multiparticulates comprising azithromycin, a pharmaceutically acceptable carrier having a melting point that is less than a melting point of said azithromycin, and a pharmaceutically acceptable dissolution enhancer, wherein said dissolution enhancer comprises a surfactant and has a concentration of acid and ester substituents of less than or equal to 0.13 meq/g azithromycin, wherein the concentration of azithromycin esters in said composition is less than about 1 wt % and wherein said azithromycin is at least 70% crystalline.

2. The composition of claim 1 wherein the concentration of azithromycin esters in said composition is less than about 0.5 wt %.

3. The composition of claim 2 wherein the concentration of azithromycin esters is less than about 0.2 wt %.

4. The composition of claim 3 wherein the concentration of azithromycin esters is less than about 0.1 wt %.

5. The composition of claim 1 wherein said azithromycin is at least 80% crystalline.

6. The composition of claim 1 wherein said azithromycin is at least 90% crystalline.

7. The composition of claim 1 wherein said dissolution enhancer comprises less than 30 wt % of said multiparticulate.

8. The composition of claim 1 wherein said dissolution enhancer is selected from the group consisting of poloxamers, polysorbates, polyoxyethylene alkyl esters, polyoxyethylene alkyl ethers, polyoxyethylene castor oil derivatives, polyoxyethylene sorbitan fatty acid esters, sorbitan esters, sodium lauryl sulfate and mixtures thereof.

9. The composition of claim 1 wherein said carrier is selected from the group consisting of non-reactive carriers, low reactivity carriers, and moderate reactivity carriers.

10. The composition of claim 9 wherein said carrier is selected from the group consisting of waxes, glycerides, and mixtures thereof.

11. The composition of claim 10 wherein said carrier is selected from the group consisting of synthetic wax, microcrystalline wax, paraffin wax, carnauba wax, glyceryl monooleate, glyceryl monostearate, glyceryl palmitostearate, polyethoxylated castor oil derivatives, hydrogenated vegetable oils, glyceryl mono-, di- and tribehenates, glyceryl tristearate, glyceryl tripalmitate and mixtures thereof.

12. The composition of claim 1 wherein said azithromycin is at least 80% crystalline.

13. The composition of any of claim 1 wherein said azithromycin is in the form of the crystalline dihydrate.

14. The composition of claim 1 wherein said multiparticulates are prepared by a melt-congeal processes.

15. The composition of claim 1 wherein said multiparticulates comprise from about 20 to about 75 wt % of said azithromycin, from about 25 to about 80 wt % of said carrier, and from about 0.1 to about 30 wt % of said dissolution enhancer.

16. The composition of claim 15 wherein said multiparticulates comprise from about 35 to about 55 wt % of said azithromycin, from about 40 to about 65 wt % of said carrier, and from about 0.1 to about 15 wt % of said dissolution enhancer.

17. The composition of claim 16 wherein said multiparticulates comprise from about 45 to about 55 wt % azithromycin, and from about 45 to about 55 wt % of said carrier.

18. The composition of claim 17 wherein said dissolution enhancer is selected from the group consisting of poloxamers, polysorbates, polyoxyethylene alkyl esters, polyoxyethylene alkyl ethers, polyoxyethylene castor oil derivatives, polyoxyethylene sorbitan fatty acid esters, sorbitan esters, sodium lauryl sulfate and mixtures thereof.

19. The composition of claim 18 wherein said dissolution enhancer is a poloxamer.

20. The composition of claim 19 wherein said carrier is a mixture of glyceryl mono-, di-, and tribehenates.

21. The composition of claim 14 wherein said azithromycin is at least 80 wt % crystalline.

22. (canceled)

23. An azithromycin dosage form for a human patient comprising a dose of from about 30 to about 90 mgA/kg of said patient's body weight of the composition of claim 1.

24. The dosage form of claim 23 wherein said dose is from about 45 to about 75 mgA/kg.

25. The dosage form of claim 24 wherein said dose is about 60 mgA/kg.

26. An azithromycin dosage form for a human patient comprising from 250 mgA to 7 gA of the composition of claim 1.

27. The dosage form of claim 26 comprising from about 1.5 to about 4 gA.

28. The dosage form of claim 27 comprising 1.8 gA to 2.2 gA.
